(19) United States
(12) Patent Application Publication
(10) Pub. No.: US 2008/0125319 A1
(43) Pub. Date: May 29, 2008

(54) Treatment of plants and plant propagation materials with an antioxidant to improve plant health and/or yield

(75) Inventors: Jawed Asrar, Chesterfield, MO (US); June E. Bourque, St. Louis, MO (US); Yiwei Ding, Ballwin, MO (US); Ernest F. Sanders, Lake St. Louis, MO (US)

Correspondence Address: Charles E. Dunlap;Nelson Mullins Riley & Scarborough, LLP, 1320 Main Street, Columbia, SC, 29201, US

(73) Assignee: Monsanto Technology, LLC, ST. LOUIS, MO (US)

(21) Appl. No.: 12/011,424

(22) Filed: Jan. 25, 2008

Related U.S. Application Data
(63) Continuation of application No. 10/832,578, filed on Apr. 27, 2004.
(60) Provisional application No. 60/466,104, filed on Apr. 28, 2003.

(51) Int. Cl. A01N 25/26; A01N 25/00; A01H 5/00

(52) U.S. Cl. 504/100; 504/116.1; 800/320.1

(57) ABSTRACT

Methods and compositions are described for the treatment of plants and plant propagation materials with an antioxidant alone or in combination with a pesticide with the result that treated plant propagation materials demonstrate improved germination rates, and plants that grow from treated plant propagation materials, or plants that are treated directly, show improved stand density or vigor, and/or improved yields.

### CROSS-REFERENCE TO RELATED PATENTS AND PATENT APPLICATIONS

The subject matter of the present application is related to and claims the benefit of U.S. Provisional Patent Application Ser. No. 60/466,104 having a filing date of Apr. 28, 2003, which is incorporated by reference herein in its entirety.

### BACKGROUND OF THE INVENTION

(1) Field of the Invention

The present invention relates to the treatment of plants and plant propagation materials for the improvement of plant health, the improvement of environmental stress tolerance, and/or the improvement in yield, and more particularly to methods and compositions for obtaining such improvements by treating plants and plant propagation materials with certain chemical compounds.

(2) Description of the Related Art

Many factors affect the quality of agricultural crops. Soil properties, fertilizers, pesticides, and labor and machinery all influence germination, emergence, vigor and yield. Environmental conditions, however, play a greater role than almost any of these. Wet, cold, drought, heat, hail and wind are all conditions that can severely reduce crop yield under certain circumstances.

A particular environmental problem that has become more important as efforts intensify to increase crop output is cold stress to planted seeds prior to and during germination. Seeds that have been chilled below temperatures that are normal for that crop after planting and prior to and during germination can suffer a significant reduction in their germination rate and an increase in the fraction of plants that are weak or malformed. Ultimately, the yield of the crop is reduced.

Increasing the cold tolerance of seeds and plants has naturally been of great interest. In fact, the ability to withstand only an additional one or two degrees of abnormal chill could provide significant improvement in crop yield. Previous attempts to improve cold hardiness have include efforts to improve the cold hardiness of plants, as well as efforts to improve the cold hardiness of plant seeds.

Early attempts to improve a plant's resistance to chilling involved altering cultivation techniques. In 1993, Nelson et al., concluded that maintaining Jojoba plants in a water-stressed condition going into the winter significantly improved the chances of frost survival (http://www.hort.purdue.edu/newcrop/proceedings1993/v2-360.html; Jul. 26, 2002).

Another technique of improving a plant's cold tolerance without biochemical intervention is that of breeding a hybrid plant from parents that demonstrate unusually high tolerances to particular stresses. In fact, there are many U.S. patents of plants that possess a unique tolerance to cold temperatures. Examples include PP9,847, PP9,982, PP9,943, PP10,030, and PP10,289, among others.

Advances in biotechnology permitted genetic manipulation for the enhancement of certain characteristics. For example, U.S. Pat. No. 5,837,545 described a DNA nucleotide sequence encoding a protein which enhanced cold tolerance of a plant cell. Other DNA encoding factors associated with cold tolerance have been identified by researchers at the University of Quebec and McGill University (See, http://abstracts.aspb.org/aspb1997/45/1250. shtml; Jul. 26, 2002).

Many studies have focused on membrane structure and function. Compounds that have been identified as being involved in increasing cold tolerance are glycerol-3-phosphate acyl transferases, chloroplastic fatty acid desaturases, reactive oxygen species, and polyamines. In particular, it was discovered that chilling tolerant plants have more efficient antioxidant systems than their chill-sensitive counterparts (http://klp.hunan.net/pas-c/pa2.pdf; Jul. 26, 2002). This was thought to be due to the fact that harsh temperatures cause plants to produce oxygen free radicals, the effects of which can be controlled by adequate antioxidants.

In 1999, researchers at the University of Arkansas performed experiments involving glycinebetaine, which resulted in support for the compound's role in the improvement of drought and heat stress (http://www.uark.edu/depts/agripub/Publications/specialreports/198su11.pdf; Jul. 26, 2002). Further experiments into the mechanisms of cold acclimation have shown that naturally produced glycinebetaine levels increase in cooler temperatures, and those plants with elevated levels of glycinebetaine are best able to adapt and survive (http://abstracts.aspb.orq/aspb1997/45/1101.shtml; Jul. 26, 2002). Furthermore, U.S. Pat. No. 6,310,271 discloses a full length choline monooxygenase (CMO) cDNA cloned and used to transform plants which do not naturally express CMO. This method improves the stress tolerance of crops that lack glycinebetaine accumulation. Similarly, U.S. Pat. Nos. 5,851,953, 5,922,649 and 5,952,267 disclose methods in which the use of glycinebetaine improves the yield of plants by reducing the adverse effects of stress.

It is not certain, however, that cold hardiness in plants also improves the cold hardiness of seeds, and less information can be found regarding the improvement of cold tolerance of seeds than is available regarding the cold hardiness of plants.

Examples of efforts involving the treatment of seeds to improve cold hardiness and drought tolerance include the following:

In 1996, Australian researchers reported that cotton seeds treated with glycinebetaine demonstrated an improvement in drought tolerance (http://life.csu.edu.au.agronomy/papers/221/221.html; Jul. 26, 2002).

Lada et al. reported the treatment of carrot seed with Ambiol™, a proprietary antioxidant and bioprotectant, glycinebetaine, and thermogenic compounds such as DHBA, acetylsalicylic acid and salicylic acid. PCPR Research Information Sheet 2000-1 (http://www.nsac.ns.ca/pas/instind/pcrp/facts.htm; Jul. 25, 2002). They reported that Ambiol™ (2-methyl-4-[dimethylaminomethyl]-5-hydroxybenzimidazole) and glycinebetaine hastened germination in carrots.

Li et al., in WO 01/82697 describe the treatment of plant seed with a composition that included at least one choline-containing compound, a calcium-containing salt, a potassium-containing salt, and salicylic acid. They reported that the composition increased the emergence and yield of soybeans and corn.

Borsos-Matovina, V. et al., Seed Treatment with the Antioxidant Ambiol Enhances Membrane Protection in Seedlings Exposed to Drought and Low Temperatures; at http://springer-ny.com/link/service/journals/oo468/content . . . /s004680000083ch002.htm; Ambiol™ at 0, 0.1, and 10 mg/l used to soak seeds of black spruce and jack pine, reduced membrane leakage.

Pierce, Synchronizing Emergence of Carrots Using Chemoembryoconditioning Agents; soaked carrot seeds in gibberellic acid (1 microgram/1 and 100 microgram/l), Ambiol™ (0.1 mg/l), and glycinebetaine (100 mg/l), and found that it accelerated emergence of seedlings.

Blake et al., Use of Seed Treatments with Antioxidants to Enhance Growth and Stress Tolerance; at http://www.forestry.utoronto.ca/ecophys/antioxid.htm; describes seed treatment with Ambiol™ and Vitamin C.

Bodapati, in Australian patent publication AU-B-27071/95 describes a method for the treatment of seeds with betaines to enhance seedling growth and/or protect against environmental stress during germination. In the method, the seed is immersed in a solution of the betaine of a concentration of 0.34 M or lower, or the seed is coated with a solid form of the betaine at a ratio of 1-10 betaine weight per seed weight.

Although many techniques have been discovered for improving the performance of plants and plant seeds, methods for improving seed germination, plant density and vigor, and/or plant product yield, are still needed. It would be particularly useful if such methods would provide these improvements when the seed or the plant is under conditions of environmental stress, such as experienced in abnormally cold or dry conditions. It would also be useful if these methods were easy and inexpensive to administer. Because some significant fraction of treated seeds are never planted, it would be particularly useful if the treatment method would not prevent the treated seeds from being used in some other fashion, such as, for example, in food, animal feed, fermentation, or the manufacture of starch, protein meal, oil, or other seed-derived product.

### SUMMARY OF THE INVENTION

Briefly, therefore the present invention is directed to a novel method of improving the health and/or yield of a plant that is grown from plant propagation material, the method comprising treating the plant or the plant propagation material with an antioxidant and a pesticide, wherein the amount of the antioxidant and the amount of the pesticide together are effective to improve the health and/or the yield of the plant.

The present invention is also directed to a novel composition for treating a plant seed, the composition comprising an antioxidant and a pesticide.

The present invention is also directed to a novel method of improving the health and/or yield of a plant that is grown from plant propagation material, the method comprising treating the plant or the plant propagation material with a composition comprising an effective amount of an antioxidant that is other than a betaine, Vitamin C, salicylic acid, acetylsalicylic acid, 2,6-dihydroxybenzoic acid (DHBA), or 2-methyl-4-[dimethylaminomethyl]-5-hydroxybenzimidazole.

The present invention is also directed to a novel method of improving the health and/or yield of a plant that is grown from a seed, the method comprising treating the plant or the plant propagation material with an anti-microbial that is selected from the group consisting of vanillin, thymol, eugenol, citral, carbacrol, biphenyl, phenyl hydroquinone, Na-o-phenylphenol, thiabendazole, K-sorbate, Na-benzoate, trihydroxybutylphenone, and propylparaben, in amount that is effective to improve the health and/or the yield of the plant.

The present invention is also directed to novel treated plant propagation material, comprising: a) plant propagation material; including, b) an antioxidant that has been added to the plant propagation material; and c) a pesticide.

The present invention is also directed to novel treated plant propagation material, comprising: a) plant propagation material; including b) an effective amount of an antioxidant that has been added to the plant propagation material and is other than a betaine, Vitamin C, salicylic acid, acetylsalicylic acid, or 2-methyl-4-[dimethylaminomethyl]-5-hydroxybenzimidazole.

The present invention is also directed to novel treated plant propagation material, comprising: a) plant propagation material; including b) an effective amount of an anti-microbial that has been added to the plant propagation material and that is selected from the group consisting of vanillin, thymol, eugenol, citral, carbacrol, biphenyl, phenyl hydroquinone, Na-o-phenylphenol, thiabendazole, K-sorbate, Na-benzoate, trihydroxybutylphenone, and propylparaben.

Among the several advantages found to be achieved by the present invention, therefore, may be noted the provision of methods and compositions for improving germination of plant propagation material, plant density and vigor, and/or plant product yield, the provision of such methods and compositions that provide these improvements when the plant propagation material or the plant is under conditions of environmental stress, such as experienced in abnormally cold or dry conditions, the provision of methods and compositions that are easy and inexpensive to administer, and, in preferred embodiments, the provision of methods and compositions that will not prevent the treated plant propagation material from being used in some other fashion, such as, for example, in food, animal feed, fermentation, or the manufacture of starch, protein meal, oil, or other plant propagation material-derived product.

### BRIEF DESCRIPTION OF THE DRAWINGS

FIG. 1A is a graph showing percent germination as a function of time at 10° C. for Hybrid A corn seeds that had received no antioxidant treatment (Control), or that had been treated with a 50/50 w/w mixture of BHA/BHT at levels of 10, 25, 50, 100 and 250 gm of antioxidant per 100 kg of seed;

FIG. 1B is a graph showing percent germination as a function of time at 10° C. for Hybrid B corn seeds that had received no antioxidant treatment (Control), or that had been treated with a 50/50 w/w mixture of BHA/BHT at levels of 10, 25, 50, 100 and 250 gm of antioxidant per 100 kg of seed;

FIG. 2A is a graph showing percent germination as a function of time at 10° C. for Hybrid A corn seeds that had received no antioxidant treatment (Control), or that had been treated with a composition containing choline chloride at levels of 10, 25, 50, 100 and 250 gm of antioxidant per 100 kg of seed;

FIG. 2B is a graph showing percent germination as a function of time at 10° C. for Hybrid B corn seeds that had received no antioxidant treatment (Control), or that had been treated with a composition containing choline chloride at levels of 10, 25, 50, 100 and 250 gm of antioxidant per 100 kg of seed;

FIG. 3A is a bar chart showing the percent germination under optimum (warm) conditions of Hybrid A corn seeds that had received no antioxidant treatment (UTC), or that had been treated with a composition containing choline chloride at levels of 10, 25, 50, 100 and 250 grams antioxidant per 100 kg of seeds;

FIG. 3B is a bar chart showing the percent germination under optimum (warm) conditions of Hybrid A corn seeds that had received no antioxidant treatment (UTC), or that had been treated with a composition containing a 50/50 w/w mixture of BHA/BHT at levels of 10, 25, 50, 100 and 250 grams antioxidant per 100 kg of seeds;

FIG. 4A is a bar chart showing the fresh weight after cold stress of soybean plants that were grown from seed that had been treated with glycinebetaine at levels of 0, 10, 20, 50, 250 and 500 gm of antioxidant per 100 kg of seed;

FIG. 4B is a bar chart showing the fresh weight after cold stress of corn plants that were grown from seed that had been treated with a composition containing chlorine chloride at levels of 0, 50 and 100 gm of antioxidant per 100 kg of seed, or a 50/50 w/w mixture of BHA/BHT at a level of 250 gm of antioxidant per 100 kg of seed;

FIG. 5A is a graph of percent emergence versus time for Hybrid A corn seeds that had received no treatment with an antioxidant (UTC), or which had been treated with a composition containing choline chloride at levels of 10, 25, or 50 gm antioxidant per 100 kg of seeds;

FIG. 5B is a graph of percent emergence versus time for Hybrid B corn seeds that had received no treatment with an antioxidant (UTC), or which had been treated with a composition containing choline chloride at levels of 10, 25, or 50 gm antioxidant per 100 kg of seeds;

FIG. 6A is a bar chart showing the effect on plant density for soybean plants that had been grown from seed having no treatment (Control AG2103), or which had been treated with silthiofam at the rate of 25 g/100 kg seed (silthiofam 25 g), or with choline chloride at 250 g/100 kg seed (CC 250 g), or with glycinebetaine at 50 g/100 kg seed (GB, 50 g), or with a 50/50 w/w mixture of BHA/BHT at 10 g/100 kg seed (BHA/BHT 10 g), or with choline chloride at 250 g/100 kg seed and a 50/50 w/w mixture of BHA/BHT at 10 g/100 kg seed (CC+BHA/BHT 250 g+10 g), or with choline chloride at 250 g/100 kg seed and silthiofam at 25 g/100 kg seed (silthiofam+CC), or with silthiofam at 25 g/100 kg seed and glycine betain at 50 g/100 kg seed (silthiofam+GB), or with silthiofam at 25 g/100 kg seed, choline chloride at 250 g/100 kg seed, and a 50/50 w/w mixture of BHA/BHT at 10 g/100 kg seed (silthiofam+CC+BHA/BHT);

FIG. 6B is a bar chart showing the effect on plant density for soybean plants that had been grown from seed having no treatment (Control AG2103), or which had been treated with silthiofam at the rate of 25 g/100 kg seed (silthiofam 25 g), or with choline chloride at 250 g/100 kg seed (CC 250 g), or with glycinebetaine at 50 g/100 kg seed (GB, 50 g), or with a 50/50 w/w mixture of BHA/BHT at 10 g/100 kg seed (BHA/BHT 10 g), or with choline chloride at 250 g/100 kg seed and a 50/50 w/w mixture of BHA/BHT at 10 g/100 kg seed (CC+BHA/BHT 250 g+10 g), or with choline chloride at 250 g/100 kg seed and silthiofam at 25 g/100 kg seed (silthiofam+CC), or with silthiofam at 25 g/100 kg seed and glycine betain at 50 g/100 kg seed (silthiofam+GB), or with silthiofam at 25 g/100 kg seed, choline chloride at 250 g/100 kg seed, and a 50/50 w/w mixture of BHA/BHT at 10 g/100 kg seed (silthiofam+CC+BHA/BHT);

FIG. 6C is a bar chart showing the effect on soybean yield (bu/acre) for soybean plants that had been grown from seed having no treatment (Control AG2103), or which had been treated with silthiofam at the rate of 25 g/100 kg seed (silthiofam 25 g), or with choline chloride at 250 g/100 kg seed (CC 250 g), or with glycinebetaine at 50 g/100 kg seed (GB, 50 g), or with a 50/50 w/w mixture of BHA/BHT at 10 g/100 kg seed (BHA/BHT 10 g), or with choline chloride at 250 g/100 kg seed and a 50/50 w/w mixture of BHA/BHT at 10 g/100 kg seed (CC+BHA/BHT 250 g+10 g), or with choline chloride at 250 g/100 kg seed and silthiofam at 25 g/100 kg seed (silthiofam+CC), or with silthiofam at 25 g/100 kg seed and glycine betain at 50 g/100 kg seed (silthiofam+GB), or with silthiofam at 25 g/100 kg seed, choline chloride at 250 g/100 kg seed, and a 50/50 w/w mixture of BHA/BHT at 10 g/100 kg seed (silthiofam+CC+BHA/BHT);

FIG. 6D is a bar chart showing the percent increase in soybean yield over untreated control for soybean plants that had been grown from seed which had been treated with a silthiofam at the rate of 25 g/100 kg seed (silthiofam 25 g), or with choline chloride at 250 g/100 kg seed (CC 250 g), or with glycinebetaine at 50 g/100 kg seed (GB, 50 g), or with a 50/50 w/w mixture of BHA/BHT at 10 g/100 kg seed (BHA/BHT 10 g), or with choline chloride at 250 g/100 kg seed and a 50/50 w/w mixture of BHA/BHT at 10 g/100 kg seed (CC+BHA/BHT 250 g+10 g), or with choline chloride at 250 g/100 kg seed and silthiofam at 25 g/100 kg seed (silthiofam+CC), or with silthiofam at 25 g/100 kg seed and glycine betain at 50 g/100 kg seed (silthiofam+GB), or with silthiofam at 25 g/100 kg seed, choline chloride at 250 g/100 kg seed, and a 50/50 w/w mixture of BHA/BHT at 10 g/100 kg seed (silthiofam+CC+BHA/BHT);

FIG. 7A is a bar chart showing the effect on plant density for corn plants that had been grown from seed having no treatment (Control), or which had been treated with silthiofam at the rate of 25 g/100 kg seed (silthiofam 25 g), or with choline chloride at 250 g/100 kg seed (CC 250 g), or with glycinebetaine at 50 g/100 kg seed (GB, 50 g), or with a 50/50 w/w mixture of BHA/BHT at 10 g/100 kg seed (BHA/BHT 10 g), or with choline chloride at 250 g/100 kg seed and a 50/50 w/w mixture of BHA/BHT at 10 g/100 kg seed (CC+BHA/BHT 250 g+10 g), or with choline chloride at 250 g/100 kg seed and silthiofam at 25 g/100 kg seed (silthiofam+CC), or with silthiofam at 25 g/100 kg seed and glycine betain at 50 g/100 kg seed (silthiofam+GB), or with silthiofam at 25 g/100 kg seed, choline chloride at 250 g/100 kg seed, and a 50/50 w/w mixture of BHA/BHT at 10 g/100 kg seed (silthiofam+CC+BHA/BHT);

FIG. 7B is a bar chart showing the effect on corn yield (bu/acre) for corn plants that had been grown from seed having no treatment (Control), or which had been treated with silthiofam at the rate of 25 g/100 kg seed (silthiofam 25 g), or with choline chloride at 250 g/100 kg seed (CC 250 g), or with glycinebetaine at 50 g/100 kg seed (GB, 50 g), or with a 50/50 w/w mixture of BHA/BHT at 10 g/100 kg seed (BHA/BHT 10 g), or with choline chloride at 250 g/100 kg seed and a 50/50 w/w mixture of BHA/BHT at 10 g/100 kg seed (CC+BHA/BHT 250 g+10 g), or with choline chloride at 250 g/100 kg seed and silthiofam at 25 g/100 kg seed (silthiofam+CC), or with silthiofam at 25 g/100 kg seed and glycine betain at 50 g/100 kg seed (silthiofam+GB), or with silthiofam at 25 g/100 kg seed, choline chloride at 250 g/100 kg seed, and a 50/50 w/w mixture of BHA/BHT at 10 g/100 kg seed (silthiofam+CC+BHA/BHT); and

FIG. 7C is a bar chart showing the percent increase in corn yield over untreated control for corn plants that had been grown from seed which had been treated with silthiofam at the rate of 25 g/100 kg seed (silthiofam 25 g), or with choline chloride at 250 g/100 kg seed (CC 250 g), or with glycinebetaine at 50 g/100 kg seed (GB, 50 g), or with a 50/50 w/w mixture of BHA/BHT at 10 g/100 kg seed (BHA/BHT 10 g), or with choline chloride at 250 g/100 kg seed and a 50/50 w/w mixture of BHA/BHT at 10 g/100 kg seed (CC+BHA/BHT 250 g+10 g), or with choline chloride at 250 g/100 kg seed and silthiofam at 25 g/100 kg seed (silthiofam+CC), or with silthiofam at 25 g/100 kg seed and glycine betain at 50 g/100 kg seed (silthiofam+GB), or with silthiofam at 25 g/100 kg seed, choline chloride at 250 g/100 kg seed, and a 50/50 w/w mixture of BHA/BHT at 10 g/100 kg seed (silthiofam+CC+BHA/BHT).

### DETAILED DESCRIPTION OF THE PREFERRED EMBODIMENTS

In accordance with the present invention, it has been discovered that the health and/or the yield of important agronomic plants can be improved by treating the plant or the plant propagation material from which the plant grows with an antioxidant and a pesticide, wherein the amount of the antioxidant and the amount of the pesticide together are effective to improve the health and/or the yield of the plant. In preferred embodiments, the improvement that is provided by the novel method is unexpectedly superior to those improvements that would normally be expected from the combination of the antioxidant and the pesticide based on the effectiveness of either the antioxidant or the pesticide alone.

In an alternative embodiment, it has been discovered that the health and/or yield of a plant that is grown from plant propagation material, such as from a seed, can be improved by treating the plant propagation material with a composition comprising an effective amount of an antioxidant that is other than a betaine, Vitamin C, salicylic acid, acetylsalicylic acid, 2,6-dihydroxybenzoic acid (DHBA), or 2-methyl-4-[dimethylaminomethyl]-5-hydroxybenzimidazole. Two or more of the antioxidants can also be used, and in preferred embodiments, the combination of two or more of the antioxidants is unexpectedly superior to those improvements that would normally be expected from the combination of the two or more antioxidants based on the effectiveness of the antioxidants used alone.

The present invention also provides compositions that can be used to practice the novel methods of treatment.

Application of the present invention to corn and soybean seeds has demonstrated improvements in the germination times and rates of the seeds under cold and wet conditions, as would be encountered in early planting, while having no deleterious effect on seeds germinated at normal temperatures. More importantly, it has also been shown that the novel methods can provide yield increases in soybeans of over 5%, even over 10%, and even up to about 30% over untreated soybeans, and yield increases in corn of over 5%, even over 10%, and even up to about 20%, or more, over corn seeds that had not received the present treatment.

In the present methods and compositions, the antioxidant can be of any type. In preferred embodiments, the antioxidant is one that has a low level of phytotoxicity. When it is said that an antioxidant has a low level of phytotoxicity, it is meant that the antioxidant is not toxic to a plant or a seed when the plant or the seed is treated with an effective amount of the antioxidant. While one having skill in the art can readily identify the level of phytotoxicity of an antioxidant, an antioxidant having a low level of phytotoxicity would be expected to not substantially reduce the emergence or the vigor of plants that emerge from seed that have been treated with the antioxidant. It is also preferred that the antioxidant that is used in the present method and compositions be one that is approved for use in food, feed, or cosmetics. Examples of such approval are approval by a regulatory body, such as the U.S. Food and Drug Administration for use in food or cosmetics, or approval by the U.S. Department of Agriculture for use in animal feed. Antioxidants that have GRAS (Generally Recognized As Safe) status are examples of preferred antioxidants. In some embodiments of the present invention, it is preferred that the antioxidant is one that is added to the seed, as opposed to an antioxidant that is a natural component of the seed. However, such preferred antioxidants can include natural antioxidants that are added to the seed during the present treatment process.

Examples of materials that can serve as the antioxidant of the present invention include: glycine, glycinebetaine, choline salts, in particular choline chloride, 2(3)-tert-butyl-4-hydroxyanisole (BHA), tert-butylhydroxyquinone (TBHQ), dilauryl thiodipropionate (DLTDP), tris(nonylphenyl))phosphite (TNPP), 2,6-dihydroxybenzoic acid (DHBA), acetylsalicylic acid (ASA), salicylic acid (SA), Irganox 1076 (Ciba Geigy), Ethanox 330 (Ethyl Corp.), Tinuvin 144 (Ciba Geigy), Ambiol (2-methyl-4-[dimethylaminomethyl]-5-hydroxybenzimidazole), propyl gallate, trihydroxybutyrophenone (THBP), thiodipropionic acid and dilauryl thiodipropionate, betaines (see, AU-B-27071/95 to Bodapati, and EO 0 493 670 A1 to Lunkenheimer et al.), amines (aromatic amines and hindered amines), methionine, cysteine, proline, mannitol, phosphites, thioesters, lecithin, gum or resin guiac, Vitamin E, polyphenols, Vitamin A, carotenoids (beta-carotene), Vitamin B, Vitamin C, tocopherols, alpha-lipoic acid, coenzyme Q10 CoQ10), grape seed extract, green tea, lutein, N-acetyl Cysteine (NAC), OPCs (pycnogenols), selenium, zinc, 2,6-di-tert-para-benzoquinone, abscisic acid, bioflavonoids, DMAE (N,N-Dimethylethanolamine, precursor of choline), metronidazole, 2-methyl-5-nitroimidazole, glyoxal, polymerized 2,2,4-trimethyl-1,2-dihydroquinoline, 2-mercaptobenzimidazol, 5-tert-butyl-4-hydroxy-2-methyl-phenyl sulfide (CAS RN 96-69-5), 4-tert-butylphenol (CAS RN 98-54-4), catechol (CAS RN 120-80-9), 2-naphthol (2-hydroxynaphthalene) (CAS RN 135-19-3), octadecyl-3-(3′,5′-di-tert-butyl-4-hydroxyphenyl)propionate (CAS RN 2082-79-3), 1,3,5-trimethyl-2,4,6-tris(3,5-di-tert-butyl-4-hydroxybenzyl)benzene (CAS RN 1709-70-2), and tris-(2,4,-di-tert-butylphenyl)phosphite (CAS RN 31570-04-4).

In some embodiments, hindered phenol antioxidants are preferred. Examples of hindered phenol antioxidants include: 2,6-di-tert-butyl-p-cresol (BHT) (CAS RN 128-37-0), 2(3)-tert-butyl-4-hydroxyanisole (BHA), isobutylenated methylstyrenated phenol (CAS RN 68457-74-9), styrenated phenol (CAS RN 61788-44-1), 2,6-di-tert-butyl-4-(octadecanoxycarbonylethyl)phenol (CAS RN 2082-79-3), 4,4′-thiobis-6-(t-butyl-m-cresol) (CAS RN 96-69-5), 4,4′-butylidenebis(6-t-butyl-m-cresol) (CAS RN 85-60-9), 4,4′-(1-methylethylidene)bis[2-(1,1-dimethylethyl)]phenol (CAS RN 79-96-9), 2,2′-methylenebis(4-methyl-6-nonyl)phenol (CAS RN 7786-17-6), 4-methyl-phenol reaction products with dicyclopentadiene and isobutylene (CAS RN 68610-51-5), tetrakis-(methylene-(3,5-di-tertbutyl-4-hydrocinnamate)methane (CAS RN 6683-19-8), tert-butylhydroxyquinone (TBHQ), Irganox 1076, Ethanox 330, and 1,3,5-tris(3,5-di-tert-butyl-4-hydroxybenzyl-)-1,3,5-triazine-2,4,6(1H,3H,5H)-trione (CAS RN 27676-62-6).

In some embodiments, the use of a hindered phenol antioxidant is advantageous due to characteristics such as being generally non-staining, non-discoloring, and non-migratory (having low volatility, (all below 0.1 mm Hg at 25° C., all but one below 0.025), and low water solubility). Hindered phenol antioxidants generally have low aquatic toxicity, (all below 59 mg/l at 20° C., all but one below 0.4), and low acute mammalian toxicity. Their use is cost effective, they have low biodegradability, but are rapidly photodegraded, and typically partition to soil and sediments rather than to air or water (all have partition coefficient of 4 or higher).

A preferred class of antioxidants comprises betaines. In addition to the compound betaine, the terms “a betaine” include such compounds as glycinebetaine, beta-alaninebetaine, 2-trimethylamino-6-ketoheptanoate, prolinebetaine, proline, N-methyl-L-proline, trans-4-hydroxy-N-methyl-L-proline, cis-3-hydroxy-N-methyl-L-proline, (−)4-hydroxyprolinebetaine, histadinebetaine, tryptophanbetaine, 2-mercaptohistidine-betaine, pipecolatebetaine, and nicotinic acid betaine, as described in WO 95/35022.

Compounds that are known as compatible solutes can act as the antioxidant of the present invention. Such compounds are described by Iba in Annu. Rev. Plant Biol, 53:225-245 (2002), and comprise highly soluble organic compounds that accumulate in plants when the plants are exposed to salt, drought, and low-temperature stress, but which are not easily metabolized by the plant. Examples of compatible solutes include mannitol and other sugar alcohols, amino acids such as proline, and amino acid derivatives such as glycinebetaine.

In some embodiments, it is preferred that the antioxidant be one that is suitable for use in foods, feeds or cosmetics, as discussed above. Examples of such antioxidants include:

(a) 4-t-butylcatechol (CAS RN 98-29-3), 4,4′-biphenol (CAS RN 92-88-6), dodecyl gallate (lauryl gallate) (CAS RN 1166-52-5), propyl gallate, tert-butyl hydroquinone (TBHQ), trihydroxybutylphenone (THBP), thiodipropionic acid, dilauryl thiodipropionate, ditridecyl thiodipropionate, dimyristyl thiodipropionate, distearyl thiodipropionate, glycine, butylated hydroxy anisole (BHA), ascorbic acid (Vitamin C), butylated hydroxy toluene (BHT), retinyl acetate, retinyl palmitate, beta carotene, tocopherols (Vitamin E), trisodium and tetra sodium edetate (EDTA), lecithin, and gum or resin guiac;

(b) Gamma oryzanol antioxidants, such as, cycloartenol trans-ferulate, cycloartenol cis-ferulate, cycloartanol trans-ferulate, cycloartanol cis-ferulate, cycloeucalenol trans-ferulate, cycloeucalenol cis-ferulate, 24-methylenecycloartanol trans-ferulate, 24-methylenecycloartanol cis-ferulate, 24-methylcholesterol trans-ferulate, 24-methylcholesterol cis-ferulate, beta-sitosterol trans-ferulate, beta-sitosterol cis-ferulate, beta-sitostenol trans-ferulate, beta-sitostenol cis-ferulate, stigmasterol trans-ferulate, stigmasterol cis-ferulate, stigmastenol trans-ferulate, stigmastenol cis-ferulate, campesterol trans-ferulate, and campesterol cis-ferulate;

(c) Tocopherols and tocotrienols, such as alpha-tocopherol, beta-tocopherol, gamma-tocopherol, delta-tocopherol, alpha-tocotrienol, beta-tocotrienol, gamma-tocotrienol, delta-tocotrienol, desmethyl-tocotrienol, and didesmethyl tocotrienol;

(d) Polyphenols, such as ferulic acid, alpha-lipoic acid, methyl ferulate, rho-coumaric acid, rho-sinapic acid, and Isovitexin;

(e) Proanthocyanidins;

(f) Metal chelators, such as magnesium, calcium, and phosphorous;

(g) Carotenoids, such as, alpha-carotene, beta-carotene, lycopene, lutein, and zeaxanthine;

(h) Antioxidant enzymes, such as, glutathione peroxidase, methionine reductase, superoxide dismutase, polyphenol oxidase, catalase, coenzyme 010, aspartate amino transferase, isozyme MT-1, and isozyme AAT-2;

(i) Phytosterols, such as beta-sitosterol, campesterol, stigmasterol, sitostenol, delta(5)-avinasterol, delta(7)-stigmastenol, sterol glucoside, acylsterol glucoside, oligoglycosylsterol, monoglycosylsterol, cellotetraosylsitosterol, methylsterol, dimethylsterol, gramisterol, isofucosterol, obtusifoliol, branosterol, 28-homotyphasterol, 28-homosteasteronic acids, 6-deoxycastasterone, and beta-amyrin;

(j) Antioxidant amino acids, such as tryptophan, histidine, methionine, cystein, cystine, cysteine, proline, and arginine;

(k) B-Vitamins, such as thiamin, riboflavin, niacin, pantothenic acid, pyridoxine, betaine, dimethyl glycine, inositol, biotin, choline, and folic acid;

(l) Phytates;

(m) Polysaccharide antioxidants, such as cycloartenol-ferulic acid glycoside, diferulic acid complex, diferulic acid-calcium complex, hemicelluloses, arabinogalactan, arabinoxylan, xyloglucan, proteoglycan, glycoprotein, and arabinofuranoside;

(n) Phospholipids, such as phosphatidylserine, phosphatidylcholine, phosphatidylethanolamine, lysophophatidylcholine, and lysophosphatidylethanolamine; and

(o) Sugar alcohols, such as mannitol.

In one embodiment, the present method is carried out by treating a seed with a composition comprising an effective amount of an antioxidant that is other than a betaine, Vitamin C, salicylic acid, acetylsalicylic acid, 2,6-dihydroxybenzoic acid (DHBA), or 2-methyl-4-[dimethylaminomethyl]-5-hydroxybenzimidazole, except that if the antioxidant comprises a choline salt, the composition is free of at least one of the materials selected from the group consisting of a calcium-containing salt, a potassium-containing salt, and salicylic acid.

When it is said that the present invention “improves plant health”, those terms are meant to include the improvement of any one or more of parameters such as the germination rate of seeds, the percent emergence of the sprouted seed from the soil, the stand density, the vigor rating of the plant, or any other measure of plant health that would be recognized by one having skill in the art of plant culture. As the terms “improves” or “improvement” are used herein, what is meant is that a measured plant health parameter of a seed that has been treated with a method or composition of the present invention is improved, or better than, that same parameter as measured for a seed that has been germinated and grown under conditions that are similar to those used for the treated seed, but which seed has not received the present treatment method or compositions.

The terms “improves yield”, means any improvement in the yield of any measured plant product. For example, an increase in the bu/acre yield of soybeans or corn derived from a crop having the present treatment as compared with the bu/acre yield from untreated soybeans or corn cultivated under the same conditions would be considered to be an improved yield.

It is believed that the subject method can be used on the seed of any plant. However, it is preferably used on seeds of plant species that are agronomically important. In particular, the seeds can be of corn, peanut, canola/rapeseed, soybean, curcubits, cotton, rice, sorghum, sugar beet, wheat, barley, rye, sunflower, tomato, sugarcane, tobacco, oats, potato, as well as other vegetable and leaf crops. It is preferred that the seed is a corn, soybean, or cotton seed, and more preferred that the seed is a soybean seed.

The terms “plant propagation material” refers to biological material from which a plant can grow. Such materials include seeds, tubers, roots, meristem tissue, and the like. Seeds are preferred plant propagation materials.

Plants and seeds on which the present invention can be used can be plants and seeds that do not have a transgenic event, or can be transgenic plants and seeds. In one embodiment, it is preferred that the plant or the seed have a transgenic event that provides the plant that grows from the seed with a resistance to a herbicide. In preferred embodiments, the herbicide is a glyphosate, and the transgenic event provides resistance to glyphosate herbicides. An example of commercially available seed having glyphosate resistance are those available under the tradename Roundup Ready® from Monsanto Company, St. Louis, Mo. Also preferred are seeds and plants having a transgenic event giving resistance to imidazolinone (IMI) herbicide that are available under the tradename YieldGuard® from Monsanto Company.

Although the present method can be applied to a seed at any state of development, it is preferred that the method is applied after the seed has been harvested and before the seed has been planted. It is also preferred that the subject method be applied to a seed that has been dried to a moisture level that is suitable for stable storage.

The pesticide of the present invention can be selected from herbicides, molluscicides, insecticides, nematocides, acaricides, fungicides, bactericides, anti-microbials, and the like. The present method is useful when the plant or the plant propagation material is treated with only one type of pesticide, but it is also useful when more than one type of pesticide is used. For example, the plant or the plant propagation material can be treated with a fungicide and one or more insecticides, or any such combination.

In some instances, compounds that have antioxidant activity can also act as pesticides. Such compounds are included in the invention and their application to a seed can satisfy the requirement for treatment of the plant or the plant propagation material with an antioxidant and a pesticide. In preferred embodiments, the pesticide that is used in the present invention is different than the antioxidant. That is, the pesticide and the antioxidant are not the same chemical.

Pesticides suitable for use in the invention include pyrethrins and synthetic pyrethroids; azoles, oxadizine derivatives; chloronicotinyls; nitroguanidine derivatives; triazoles; organophosphates; pyrrols; pyrazoles; phenyl pyrazoles; diacylhydrazines; biological/fermentation products; and carbamates. Examples of pesticides within these categories are listed in The Pesticide Manual, 12th Ed., C. D. S. Tomlin, Ed., British Crop Protection Council, Farnham, Surry, UK (2000).

Pyrethroids that are useful in the present composition include pyrethrins and synthetic pyrethroids. Examples of pyrethrins that are preferred for use in the present method include, without limitation, 2-allyl-4-hydroxy-3-methyl-2-cyclopenten-1-one ester of 2,2-dimethyl-3-(2-methylpropenyl)-cyclopropane carboxylic acid, and/or (2-methyl-1-propenyl)-2-methoxy-4-oxo-3-(2-propenyl)-2-cyclopenten-1-yl ester and mixtures of cis and trans isomers thereof (Chemical Abstracts Service Registry Number (“CAS RN”) 8003-34-7).

Examples of synthetic pyrethroids that are preferred for use in the present invention include (s)-cyano(3-phenoxyphenyl)methyl-4-chloro alpha (1-methylethyl)benzeneacetate (fenvalerate, CAS RN 51630-58-1), (S)-cyano-(3-phenoxyphenyl)methyl (S)-4-chloro-alpha-(1-methylethyl)benzeneacetate (esfenvalerate, CAS RN 66230-04-4), (3-phenoxyphenyl)-methyl(+)cis-trans-3-(2,2-dichoroethenyl)-2,2-dimethylcyclopropanecarboxylate (permethrin, CAS RN 52645-53-1), (±) alpha-cyano-(3-phenoxyphenyl)methyl(+)-cis,trans-3-(2,2-dichloroethenyl)-2,2-dimethyl-cyclopropane carboxylate (cypermethrin, CAS RN 52315-07-8), (beta-cypermethrin, CAS RN 65731-84-2), (theta cypermethrin, CAS RN 71697-59-1), S-cyano (3-phenoxyphenyl)methyl (±) cis/trans 3-(2,2-dichloroethenyl) 2,2 dimethylcyclopropane carboxylate (zeta-cypermethrin, CAS RN 52315-07-8), (s)-alpha-cyano-3-phenoxybenzyl (1R,3R)-3-(2,2-dibromovinyl)-2,2-dimethylcyclopropanecarboxylate (deltamethrin, CAS RN 52918-63-5), alpha-cyano-3-phenoxybenzyl 2,2,3,3,-tetramethyl cyclopropoanecarboxylate (fenpropathrin, CAS RN 64257-84-7), (RS)-alpha-cyano-3-phenoxybenzyl(R)-2-[2-chloro-4-(trifluoromethyl)anilino]-3-methylbutanoate (tau-fluvalinate, CAS RN 102851-06-9), (2,3,5,6-tetrafluoro-4-methylphenyl)methyl-(1-alpha, 3-alpha)-(Z)-(±)-3-(2-chloro-3,3,3-trifluoro-1-propenyl)-2,2-dimethylcyclopropanecarboxylate (tefluthrin, CAS RN 79538-32-2), (±)-cyano (3-phenoxyphenyl)methyl (±)-4-(difluoromethoxy)-alpha-(1-methyl ethyl)benzeneacetate (flucythrinate, CAS RN 70124-77-5), cyano(4-fluoro-3-phenoxyphenyl)methyl 3-[2-chloro-2-(4-chlorophenyl)ethenyl]-2,2-dimethylcyclopropanecarboxylate (flumethrin, CAS RN 69770-45-2), cyano(4-fluoro-3-phenoxyphenyl) methyl 3-(2,2-dichloroethenyl)-2,2-dimethyl-cyclopropanedarboxylate (cyfluthrin, CAS RN 68359-37-5), (beta cyfluthrin, CAS RN 68359-37-5), (transfluthrin, CAS RN 118712-89-3), (S)-alpha-cyano-3-phenoxybenzyl(Z)-(1R-cis)-2,2-dimethyl-3-[2-(2,2,2-trifluoro-trifluoromethyl-ethoxycarbonyl)vinyl]cyclopropane carboxylate (acrinathrin, CAS RN 101007-06-1), (1R cis) S and (1S cis) R enantiomer isomer pair of alpha-cyano-3-phenoxybenzyl-3-(2,2-dichlorovinyl)-2,2-dimethylcyclopropane carboxylate (alpha-cypermethrin, CAS RN 67375-30-8), [1R,3S)3(1′RS)(1′,2′,2′,2′-tetrabromoethyl)]-2,2-dimethylcyclopropanecarboxylic acid (s)-alpha-cyano-3-phenoxybenzyl ester (tralomethrin, CAS RN 66841-25-6), cyano-(3-phenoxyphenyl)methyl 2,2-dichloro-1-(4-ethoxyphenyl)cyclopropane carboxylate (cycloprothrin, CAS RN 63935-38-6), [1α,3α(Z)]-(±)-cyano-(3-phenoxyphenyl)methyl 3-(2-chloro-3,3,3-trifluoro-1-propenyl)-2,2-dimethylcyclopropanecarboxylate (cyhalothrin, CAS RN 68085-85-8), [1-alpha (s), 3-alpha(z)]-cyano(3-phenoxyphenyl)methyl-3-(2-chloro-3,3,3-trifluoro-1-propenyl)-2,2-dimethylcyclopropanecarboxylate (lambda cyhalothrin, CAS RN 91465-08-6), (2-methyl-[1,1′-biphenyl]-3-yl)methyl-3-(2-chloro-3,3,3-trifluoro-1-propenyl)-2,2-dimethyl-cyclopropanecarboxylate (bifenthrin, CAS RN 82657-04-3), 5-1-benzyl-3-furylmethyl-d-cis(1R,3S, E)2,2-dimethyl-3-(2-oxo,-2,2,4,5 tetrahydro thiophenylidenemethyl)cyclopropanecarboxylate (kadethrin, RU 15525, CAS RN 58769-20-3), [5-(phenylmethyl)-3-furanyl]-3-furanyl-2,2-dimethyl-3-(2-methyl-1-propenyl)cyclopropane carboxylate (resmethrin, CAS RN 10453-86-8), (1R-trans)-[5-(phenylmethyl)-3-furanyl]methyl 2,2-dimethyl-3-(2-methyl-1-propenyl)cyclopropanecarboxylate (bioresmethrin, CAS RN 28434-01-7), 3,4,5,6-tetrahydro-phthalimidomethyl-(1RS)-cis-trans-chrysanthemate (tetramethrin, CAS RN 7696-12-0), 3-phenoxybenzyl-d,l-cis,trans 2,2-dimethyl-3-(2-methylpropenyl)cyclopropane carboxylate (phenothrin, CAS RN 26002-80-2); (empenthrin, CAS RN 54406-48-3); (cyphenothrin; CAS RN 39515-40-7), (prallethrin, CAS RN 23031-36-9), (imiprothrin, CAS RN 72963-72-5), (RS)-3-allyl-2-methyl-4-oxcyclopent-2-enyl-(1S,3R; 1R,3S)-2,2-dimethyl-3-(2-methylprop-1-enyl)cyclopropanecarboxylate (allethrin, CAS RN 584-79-2), (bioallethrin, CAS RN 584-79-2), and (ZXI8901, CAS RN 160791-64-0). It is believed that mixtures of one or more of the aforementioned synthetic pyrethroids can also be used in the present invention. Particularly preferred synthetic pyrethroids are tefluthrin, lambda cyhalothrin, bifenthrin, permethrin and cyfluthrin. Even more preferred synthetic pyrethroids are tefluthrin and lambda cyhalothrin, and yet more preferred is tefluthrin.

Oxadiazine derivative insecticides are useful in the subject method. Examples of oxadizine derivative insecticides that are preferred for use in the present invention are those that are identified in U.S. Pat. No. 5,852,012. More preferred oxadiazine derivatives are 5-(2-chloropyrid-5-ylmethyl)-3-methyl-4-nitroiminoperhydro-1,3,5-oxadiazine, 5-(2-chlorothiazol-5-ylmethyl)-3-methyl-4-nitroiminoperhydro-1,3,5-oxadiazine, 3-methyl-4-nitroimino-5-(1-oxido-3-pyridinomethyl)perhydro-1,3,5-oxadiazine, 5-(2-chloro-1-oxido-5-pyridiniomethyl)-3-methyl-4-nitroiminoperhydro-1,3,5-oxidiazine; and 3-methyl-5-(2-methylpyrid-5-ylmethyl)-4-nitroiminoperhydro-1,3,5-oxadiazine. Even more preferred is thiamethoxam (CAS RN 153719-23-4).

Chloronicotinyl insecticides are also useful in the subject method. Examples of chloronicotinyls that are preferred for use in the subject composition are described in U.S. Pat. No. 5,952,358, and include acetamiprid ((E)-N-[(6-chloro-3-pyridinyl)methyl]-N′-cyano-N-methyleneimidamide, CAS RN 135410-20-7), imidacloprid (1-[(6-chloro-3-pyridinyl)methyl]-N-nitro-2-imidazolidinimime, CAS RN 138261-41-3), and nitenpyram (N-[(6-chloro-3-pyridinyl)methyl]-N-ethyl-N′-methyl-2-nitro-1,1-ethenediamine, CAS RN 120738-89-8).

Nitroguanidine insecticides are useful in the present method. Such nitroguanidines include those described in U.S. Pat. Nos. 5,633,375, 5,034,404 and 5,245,040, and, in particular, TI-435 (N-[(2-chloro-5-thiazoyl)methyl]-N′-methyl-N″-nitro[C(E)]-(9Cl)-guanidine, (having a common name of clothianidin) CAS RN 210880-92-5), and dinotefuran (N-methyl-N′-nitro-N″-[(tetrahydro-3-furanyl)methyl)]guanidine; GAS RN 165252-70-0).

In some nomenclatures, chloronicotinyl insecticides, nitroguanidine insecticides, and some oxadiazine insecticides are referred to together as neonicotinoids.

Pyrrols, pyrazoles and phenyl pyrazoles that are useful in the present method include those that are described in U.S. Pat. No. 5,952,358. Preferred pyrazoles include chlorfenapyr (4-bromo-2-(4-chlorophenyl)-1-ethoxymethyl-5-trifluoromethylpyrrole-3-carbonitrile, CAS RN 122453-73-0), fenpyroximate ((E)-1,1-dimethylethyl-4[[[[(1,3-dimethyl-5-phenoxy-1H-pyrazole-4-yl)methylene]amino]oxy]methyl]benzoate, CAS RN 111812-58-9), and tebufenpyrad (4-chloro-N[[4-1,1-dimethylethyl)phenyl]methyl]-3-ethyl-1-methyl-1H-pyrazole-5-carboxamide, CAS RN 119168-77-3). A preferred phenyl pyrazole is fipronil (5-amino-[2,6-dichloro-4-(trifluoromethyl)phenyl]-4-[(1R,S)-(trifluoromethyl)sulfinyl]-1H-pyrazole-3-carbonitrile, CAS RN 120068-37-3).

Diacylhydrazines that are useful in the present invention include halofenozide (4-chlorobenzoate-2-benzoyl-2-(1,1-dimethylethyl)-hydrazide, CAS RN 112226-61-6), methoxyfenozide (RH-2485; N-tert-butyl-N′-(3-methoxy-o-toluoyl)-3,5-xylohydrazide, CAS RN 161050-58-4), and tebufenozide (3,5-dimethylbenzoic acid 1-(1,1-dimethylethyl)-2-(4-ethylbenzoyl)hydrazide, CAS RN 112410-23-8).

Triazoles, such as amitrole (CAS RN 61-82-5) and triazamate are useful in the method of the present invention. A preferred triazole is triazamate (ethyl-[[1-[(dimethylamino)carbonyl]-3-(1,1-dimethylethyl)-1H-1,2,4-triazol-5-yl]thio]acetate, CAS RN 112143-82-5).

Biological/fermentation products, such as avermectin (abamectin, CAS RN 71751-41-2) and spinosad (XDE-105, CAS RN 131929-60-7) are useful in the present method.

Organophosphate insecticides are also useful as one of the components of the present method. Preferred organophophate insecticides include acephate (CAS RN 30560-19-1), chlorpyrifos (CAS RN 2921-88-2), chlorpyrifos-methyl (CAS RN 5598-13-0), diazinon (CAS RN 333-41-5), fenamiphos (CAS RN 22224-92-6), and malathion (CAS RN 121-75-5).

In addition, carbamate insecticides are useful in the subject method. Preferred carbamate insecticides are aldicarb (CAS RN 116-06-3), carbaryl (CAS RN 63-25-2), carbofuran (CAS RN 1563-66-2), oxamyl (CAS RN 23135-22-0) and thiodicarb (CAS RN 59669-26-0).

Fungicides that are useful in the present invention include tebuconazole, simeconazole, fludioxonil, fluquinconazole, difenoconazole, 4,5-dimethyl-N-(2-propenyl)-2-(trimethylsilyl)-3-thiophenecarboxamide (silthiopham), hexaconazole, etaconazole, propiconazole, triticonazole, flutriafol, epoxiconazole, fenbuconazole, bromuconazole, penconazole, imazalil, tetraconazole, flusilazole, metconazole, diniconazole, myclobutanil, triadimenol, bitertanol, pyremethanil, cyprodinil, tridemorph, fenpropimorph, kresoxim-methyl, azoxystrobin, ZEN90160, fenpiclonil, benalaxyl, furalaxyl, metalaxyl, R-metalaxyl, orfurace, oxadixyl, carboxin, prochloraz, trifulmizole, pyrifenox, acibenzolar-5-methyl, chlorothalonil, cymoaxnil, dimethomorph, famoxadone, quinoxyfen, fenpropidine, spiroxamine, triazoxide, BAS50001F, hymexazole, pencycuron, fenamidone, guazatine, and cyproconazole.

Anti-microbials that are useful in the invention vanillin, thymol, eugenol, citral, carbacrol, biphenyl, phenyl hydroquinone, Na-o-phenylphenol, thiabendazole, K-sorbate, Na-benzoate, trihydroxybutylphenone, and propylparaben.

Herbicides that are useful in the present invention include acetochlor, acifluorfen, aclonifen, acrolein, AKH-7088, alachlor, alloxydim, ametryn, amicarbazone, amidosulfuron, amitrole, ammonium sulfamate, anilofos, asulam, atrazine, azafenidin, azimsulfuron, BAS 625H, beflubutamid, benazolin, benfluralin, benfuresate, bensulfuron-methyl, bensulide, bentazone, benzobicyclon, benzofenap, bifenox, bilanafos, bispyribac-sodium, borax, bromacil, bromobutide, bromoxynil, butachlor, butafenacil, butamifos, butralin, butroxydim, butylate, cafenstrole, carbetamide, carfentrazone-ethyl, chloramben, chlorbromuron, chlorflurenol-methyl, chloridazon, chlorimuron-ethyl, chloroacetic acid, chlorotoluron, chlorpropham, chlorsulfuron, chlorthal-dimethyl, chlorthiamid, cinidon-ethyl, cinmethylin, cinosulfuron, clethodim, clodinafop-propargyl, clomazone, clomeprop, clopyralid, cloransulam-methyl, cumyluron, cyanazine, cycloate, cyclosulfamuron, cycloxydim, cyhalofop-butyl, 2,4-D, daimuron, dalapon, dazomet, 2,4-DB, desmedipham, dicamba, dichlobenil, dichlorprop, dichlorprop-P, diclofop-methyl, diclosulam, difenzoquat metilsulfate, diflufenican, diflufenzopyr, dimefuron, dimepiperate, dimethachlor, dimethametryn, dimethenamid, dimethipin, dimethylarsinic acid, dinitramine, dinoterb, diphenamid, diquat dibromide, dithiopyr, diuron, DNOC, endothal, EPTC, esprocarb, ethalfluralin, ethametsulfuron-methyl, ethofumesate, ethoxysulfuron, etobenzanid, fenoxaprop-P-ethyl, fentrazamide, fenuron, ferrous sulfate, flamprop-M, flazasulfuron, florasulam, fluazifop-butyl, fluazifop-P-butyl, fluazolate, flucarbazone-sodium, fluchloralin, flufenacet, flumetsulam, flumiclorac-pentyl, flumioxazin, fluometuron, fluoroglycofen-ethyl, flupropanate, flupyrsulfuron-methyl-sodium, flurenol, fluridone, fluorochloridone, fluoroxypyr, flurtamone, fluthiacet-methyl, fomesafen, fosamine, glufosinate-ammonium, glyphosate, halosulfuron-methyl, haloxyfop, HC-252, hexazinone, imazamethabenz-methyl, imazamox, imazapic, imazapyr, imazaquin, imazethapyr, imazosulfuron, indanofan, iodosulfuron-methyl-sodium, ioxynil, isoproturon, isouron, isoxaben, isoxaflutole, lactofen, lenacil, linuron, MCPA, MCPA-thioethyl, MCPB, mecoprop, mecoprop-P, mefenacet, mefluidide, mesotrione, metam, metamitron, metazachlor, methabenzthiazuron, methylarsonic acid, methyldymron, methyl isothiocyanate, metobenzuron, metobromuron, metolachlor, S-metolachlor, metosulam, metoxuron, metribuzin, metsulfuron-methyl, MK-616, MKH 6561, molinate, monolinuron, naproanilide, napropamide, naptalam, neburon, nicosulfuron, nonanoic acid, norflurazon, oleic acid (fatty acids), orbencarb, oryzalin, oxadiargyl, oxadiazon, oxasulfuron, oxaziclomefone, oxyfluorfen, paraquat dichloride, pebulate, pendimethalin, pentachlorophenol, pentanochlor, pentoxazone, petroleum oils, phenmedipham, picloram, picolinafen, piperophos, pretilachlor, primisulfuron-methyl, prodiamine, prometon, prometryn, propachlor, propanil, propaquizafop, propazine, propham, propisochlor, propyzamide, prosulfocarb, prosulfuron, pyraflufen-ethyl, pyrazolynate, pyrazosulfuron-ethyl, pyrazoxyfen, pyribenzoxim, pyributicarb, pyridate, pyriminobac-methyl, pyrithiobac-sodium, quinclorac, quinmerac, quinoclamine, quizalofop, quizalofop-P, rimsulfuron, sethoxydim, siduron, simazine, simetryn, sodium chlorate, sulcotrione, sulfentrazone, sulfometuron-methyl, sulfosulfuron, sulfuric acid, tar oils, 2,3,6-TBA, TCA-sodium, tebutam, tebuthiuron, tepraloxydim, terbacil, terbumeton, terbuthylazine, terbutryn, thenylchlor, thiazopyr, thifensulfuron-methyl, thiobencarb, tiocarbazil, tralkoxydim, tri-allate, triasulfuron, triaziflam, tribenuron-methyl, triclopyr, trietazine, trifluralin, triflusulfuron-methyl, and vernolate.

Another embodiment of the invention includes a method of improving the health and/or yield of a plant that is grown from plant propapation material by treating the plant or the plant propagation material with an anti-microbial that is selected from the group described above, in an amount that is effective to improve the health and/or the yield of the plant.

When a pesticide is described herein, it is to be understood that the description is intended to include salt forms of the pesticide as well as any isomeric and/or tautomeric form of the pesticide that exhibits the same activity as the form of the pesticide that is described.

The pesticides that are useful in the present method can be of any grade or purity that pass in the trade as such pesticide. Other materials that accompany the pesticides in commercial preparations as impurities can be tolerated in the subject methods and compositions, as long as such other materials do not destabilize the composition or significantly reduce or destroy the activity of any of the pesticide components against a target pest(s). One of ordinary skill in the art of the production of pesticides can readily identify those impurities that can be tolerated and those that cannot.

The pesticides and antioxidants that are useful in the present invention can be provided in solid or liquid form, and can be provided as emulsions, dispersions, solutions, or in particulate form. They can be supplied and used alone (“neat”) or can be in combination with other materials, as long as such other materials do not destabilize, or significantly reduce or destroy the activity of the pesticide or the antioxidant.

The present method can be applied to seeds that have already been treated by others, such as commercially available seeds that have been treated with one or more pesticides, growth factors, colorants, or other agents. However, the novel method also includes the treatment of seeds that have received no prior treatment. Whenever a seed is treated with the novel method, it is preferred that the treatment is carried out so that an evenly distributed coating of the pesticide and/or the antioxidant is applied to the outer surface of the seed without loss of pesticide or antioxidant due to excess liquid falling off of the seed during or after the treatment. After addition of the pesticide and/or antioxidant, it is preferred that the seeds are not dried and are not agitated so long that the active material(s) is abraded off the seed due to dusting and scuffing.

In the embodiment of the present method in which a seed is treated with both a pesticide and an antioxidant, both materials can be applied to the seed simultaneously or sequentially to provide a seed that is treated with both the antioxidant and the pesticide. Alternatively, the seed can be treated with one of the agents prior to planting, and then treated with the other of the agents during or after planting.

The amount of the antioxidant that is applied to the seed varies according to the type and purity of the antioxidant that is selected. It is preferred, however, that the amount of the antioxidant be an effective amount.

When it is said that an “effective amount” of an antioxidant or a pesticide is used, what is meant is that the amount of the antioxidant or pesticide, or the combined amount of the antioxidant and the pesticide, if both are used in combination, is sufficient to result in an improvement in plant health and/or yield.

The amount of the antioxidant that is used to treat the seed can vary according to the type of antioxidant and the purity of the preparation that is available for use. In preferred embodiments, the amount of antioxidant that is applied to the seed is between about 0.1 gm of active ingredient/100 kg of seed and about 1,000 gm/100 kg of seed; more preferably between about 1 gm and about 750 gm/100 kg of seed, even more preferably between about 5 gm and about 500 gm/100 kg of seed, and yet more preferably between about 5 gm and about 400 gm/100 kg of seed.

When the method includes the application of a pesticide to the plant or the seed in combination with the antioxidant, the amount of the pesticide that is used depends upon the type and purity of the pesticide. However, it is preferred that the amount of the antioxidant and the amount of the pesticide together are effective to improve the health and/or the yield of the plant.

In preferred embodiments, the amount of pesticide that is applied to the seed is between about 0.1 gm of active ingredient/100 kg of seed and about 1,000 gm/100 kg of seed; more preferably between about 5 gm and about 600 gm/100 kg of seed, and even more preferably between about 25 gm and about 400 gm/100 kg of seed.

In the present invention, the antioxidant alone or in combination with a pesticide can also be applied to a plant. In this embodiment, a formulation comprising the pesticide and/or the antioxidant can be applied to the plant foliage by drip, spray, dusting, contact with a wet wick, drenching, or any other technique that is known in the art for the application of wet or dry formulations to plants. Such foliar application can be carried out at any time after the plant has emerged.

In a preferred embodiment, an antioxidant can be applied to a herbicide resistant transgenic plant along with the herbicide against which the plant is resistant. For example, an antioxidant can be applied along with a glyphosate herbicide to transgenic plants that are resistant to glyphosate. Another example is the application of an antioxidant along with an imidazolinone herbicide to transgenic plants that are resistant to imidazolinone.

The antioxidant and the pesticide can be applied to the plant or the plant propagation material in any form and such forms as capsule suspensions (CS), emulsifiable concentrates (EC), emulsions in oil or water (EO and EW), granules (GR), suspension concentrates (SC), soluble granules (SG), soluble concentrates (SL), soluble powders (SP), and water dispersible granules (WG) are suitable. It is preferred to apply the pesticide and/or the antioxidant to the seed in the form of a flowable liquid. The antioxidant and the pesticide can be in a true solution in the liquid, or it can be present as small droplets or particles to form a suspension, dispersion or emulsion. Since many pesticides have low water solubility, it is preferred that when water is the liquid, an aqueous dispersion, suspension, or emulsion of the pesticide be used, and that the pesticide be present in the dispersion, suspension, or emulsion in the form of small particles or droplets. As used herein, the term “suspension” will be considered to include any form of liquid containing small particles, and to include the terms dispersion and emulsion.

The particles of pesticide in the liquid suspension can be of any size that permits the suspension to be applied to the plant or the plant propagation material by any means, such as, for example, by spraying. It is preferred that the particles of pesticide in the suspension have a number average nominal size of less than about 100 microns, more preferably of less than about 50 microns, even more preferably of less than about 10 microns, and even more preferably of less than about 1 micron (be “sub-micron” in size). It is believed that the use of such small particles causes the pesticide to form a more stable and homogenous suspension—thereby allowing a more even distribution of the pesticide over the surface of the seed, and that the small particles are less subject to abrasion from the treated seed after the pesticide treatment has been applied.

The pesticide and/or the antioxidant can be applied to seed in any type of conventional seed treatment or coating equipment. Application in seed treating machines having the characteristics of a CMS seed coating machine (Vector Corporation, Marion, Iowa), or a Hege Treater (Hege Equipment Co., Colwich, Kans.) for example, have been found to be suitable. One method that has been found to be successful for applying a pesticide that is supplied as a solid to seed, is to mill the pesticide to 1-20 micron, or to sub-micron, size and then to add the small particles of the pesticide to water to form an aqueous suspension. A mill that is capable of reducing solids to fine particles, such as a Mirco-Jet Pulverizer air mill, available from Fluid Energy Processing and Equipment Company, Hatfield, Pa., can be used for the size reduction. A bowl mill, such as the Szegvari Attritor System, can also be used to compound the pesticides and/or antioxidants into useful formulations.

The concentration of the pesticide in the suspension should be low enough to permit easy handling and application of the suspension to the seed—such as by spraying—and thorough distribution of the pesticide among the seeds so that the outer surface of each seed is substantially covered. However, the concentration should be high enough that, when used in combination with the other parameters of seed treatment, to avoid the loss of pesticide from the seeds by dripping or pooling of the treating liquid suspension. Pesticide concentrations of between about 0.1% and about 50%, by weight, are useful for such suspensions, preferred are concentrations between about 0.5% and 30%, by weight, even more preferred are concentrations between about 0.6% and about 15%, and yet more preferred are concentrations of the pesticide between about 1% and 15%, by weight of the suspension. Sticking agents and dyes can also be added to the pesticide suspension to promote the adherence of the suspension to the seeds and to identify the seeds as having been treated.

A desired amount of the suspension of the pesticide is sprayed onto the seed in, for example, a CMS seed treater, over a period of time that is long enough to permit thorough distribution of the suspension over the seed, but short enough so that the treated seed do not completely dry. It is believed that if the treated seed are allowed to remain in a heated seed treater until the suspension is completely dry, the danger of loss of the pesticide by abrasion increases. When the exit temperature of the heated air circulating through the CMS machine is held to about 95° F., and the aqueous suspension contains about 1.6% by weight pesticide and 8% by weight of a sticking agent, an application time of between about 3 minutes and about 20 minutes is suitable, and an application time of between about 5 and about 15 minutes is preferred.

If desirable, the pesticide and/or the antioxidant that are used in the present method can be applied to the seed as a part of a coating. The coating can be a controlled-release coating. Furthermore, the seeds can be treated with materials other than the antioxidant and the pesticide, if desirable, in order to serve as plasticizers, emulsifiers, stabilizers, fillers, dyes, safeners, and the like. Such materials are well known in the art.

Seeds that have been treated by the subject method can be stored, handled and planted like any other seeds. Similar methods and conditions can be used as are used with any other treated, or non-treated seeds and the same handling and planting equipment can be used that is used for conventional seeds.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### General Procedures

### Method of Treating Seed with a Pesticide and/or an Antioxidant:

Corn, soybean and sorghum seed, which had been dried to a stable moisture level, were pre-weighed into separate samples of approximately 85 grams. A coating suspension was prepared by mixing water (at the rate of 440 g/100 kg of seed) at room temperature, with Vinac XX-210 (at the rate of 310 g 100 kg of seed; available from Air Products Corporation). If a pesticide or an antioxidant was to be applied to the seed, the desired amount of the agent was added to the coating suspension. If the pesticide or the antioxidant was a solid, it was pre-milled to particles of less than 10 micron nominal size prior to addition to the coating suspension. The ingredients were then mixed together and the mixture was stirred with a mechanical stirrer at medium speed at room temperature for 20 minutes. At this time the coating suspension was ready for application to the seed.

The seed selected for treatment was placed in the drum of a Hege 11 seed treatment machine (Hege Equipment Co., Inc., Colwich, Kans.), and the machine was turned on. After 7 seconds, the premixed coating suspension was applied into the bed of tumbling seed via a syringe within about one second and the bowl was permitted to continue to rotate for another 23 seconds. The machine was then turned off and the coated seeds were collected for testing or storage.

### Bowl Mill Method for Preparing Seed Treatment Formulations Containing Antioxidants:

In a typical example, Lomar D (2.96 g) was dissolved in water (181.10 g) and then BHA (25.01 g) and BHT (25.00 g) were added to the solution. The mixture was then added to the bowl of a Szegvari Attritor System (Type 01STD, Size 01, available from Union Process, Inc., Akron, Ohio), filled with 3/16″ stainless steel beads. The mixture in the bowl along with the beads was stirred for 2 hours and then Agsol EX 8 (0.30 g), Agnique DF6889 (0.20 g) and Emcol 4500 (0.90 g) were added and the mixture was stirred for another hour. Then a mixture of water (70.0 g) and Emcol 4500 (0.20 g) was added to the bowl and the product (195.36 g) in the bowl was collected. Finally 6.06 g of CF clear was added to the product and the formulation was ready for the seed treatment.

### Preparation of Betaine Seed Treatment Formulation:

30.02 g of betaine (available from Sigma-Aldrich, St. Louis, Mo.) was dissolved in water (109.71 g) and CF clear (4.77 g) was added to the solution under the stirring. The mixture became a white emulsion and was ready for the seed treatment.

### Germination Testing Under Optimum (Warm) Growth Conditions

Two different tests were utilized in our determination of optimum germination potential of corn and or soybean seeds.

Test 1—Ten seeds for each sample to be tested are placed in each of ten Petri dishes with 5 ml of distilled water and incubated at 30° C. day and 20° C. night with a 16 hour day length. Each day the Petri dishes were checked and scored for germination. Percent germination was determined as the average number of seeds which had germinated within the test period minus any abnormal seeds, divided by the total number of original seeds, times 100. A seed was considered germinated when the radical reached a length of 1 cm.

Test 2—Test 2 is much closer to an industry standard for optimum condition testing. Test seeds are placed on a moist paper towel and then covered with a second moist paper towel. The towel is then rolled into a cylinder and placed upright in a beaker containing water. The beaker is placed in an incubation chamber at 25° C. for 7 days in continuous darkness. After 7 days the number of seeds germinated are counted and converted to a percentage of seeds in the test. This test is used to determine the maximum germination potential of the seed.

### Test Utilizing Cold Germination Conditions:

This test was designed to measure the ability of seeds to germinate under adverse condition associated with high moisture and low temperature.

Test 3—This test was conducted as described above in Test 1, except that the Petri dishes were incubated at a constant temperature of 10° C. Germination rate was determined as described above in Test 1. The test length continued for 30 days as the seeds are very slow to germinate under these conditions.

### Soil Emergence Test:

This test was designed to measure the emergence of plantlets from sprouted seeds under conditions that more closely resemble those found in field conditions.

For each sample batch of seed to be tested, 10 seeds were placed on ½″ of soil medium in a test tray and covered with an additional 2″ of soil medium. Three replications were prepared for each sample batch. The soil is moistened regularly and placed in the desired temperature regime. For a cold emergence test, trays are incubated at 10° C. with 16 hours of light. The tray is monitored daily over a 30-day period and a seed is counted as emerged when the spike breaks through the surface of the soil. Percent emergence is calculated as the number of emerged plantlets divided by the total number of seeds that were planted, times 100.

### Example 1

This example illustrates the treatment of corn, soybean and sorghum seeds with antioxidants.

Corn, soybean and sorghum seed, which had been dried to a stable moisture level, were treated with antioxidants according to the rate shown in Table 1 as described in General Procedures.

TABLE 1
Seed treatment with selected antioxidants.

| | Antioxidant used in | Treatment rate |
|---|---|---|
| Sample | seed treatment^{a} | (g antioxidant/100 kg seed) |
| UTC | Untreated control | 0 |
| V | Vinac XX-210 polymer | 0 |
| | only | |
| CC10 | choline chloride | 10 |
| CC25 | choline chloride | 25 |
| CC50 | choline chloride | 50 |
| CC100 | choline chloride | 100 |
| CC250 | choline chloride | 250 |
| B10 | BHA/BHT | 10 |
| B25 | BHA/BHT | 25 |
| B50 | BHA/BHT | 50 |
| B100 | BHA/BHT | 100 |
| B250 | BHA/BHT | 250 |
| GB10 | Glycinebetaine | 10 |
| GB20 | Glycinebetaine | 20 |
| GB50 | Glycinebetaine | 50 |
| GB250 | Glycinebetaine | 250 |
| GB500 | Glycinebetaine | 500 |

Notes:
^{a}choline chloride was supplied by Sigma Aldrich, St. Louis, MO; the BHA/BHT was a 50%/50% by weight mixture of BHA and BHT; glycinebetaine was supplied by Sigma Aldrich, St. Louis, MO.

### Example 2

This example illustrates the effect of seed treatment with antioxidants on germination rates in optimum (warm) and cold condition germination tests for corn seeds with different degrees of cold tolerance.

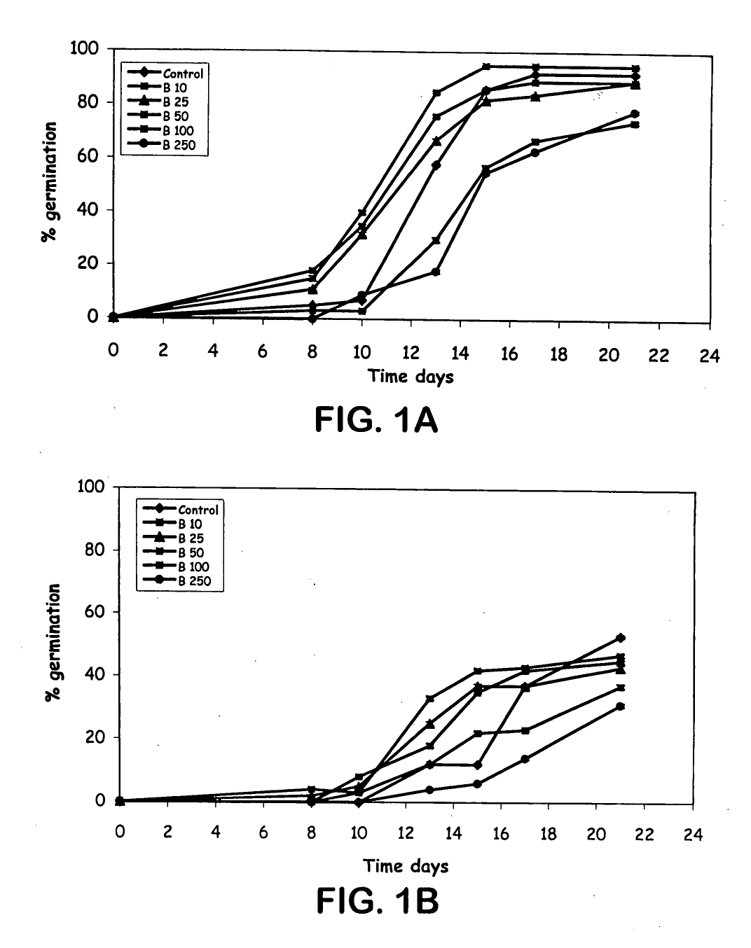

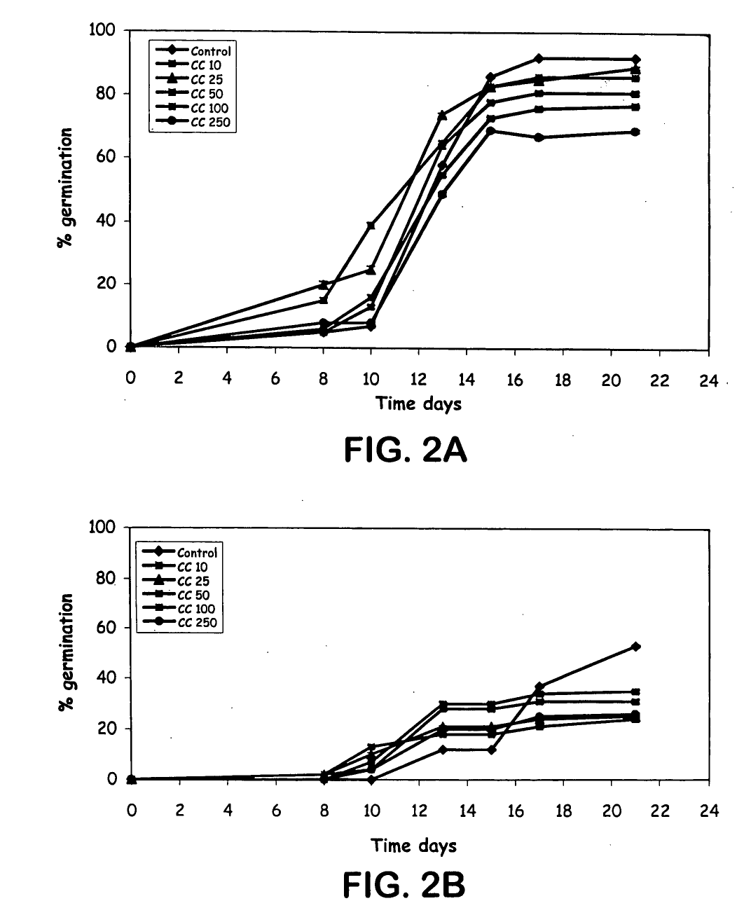

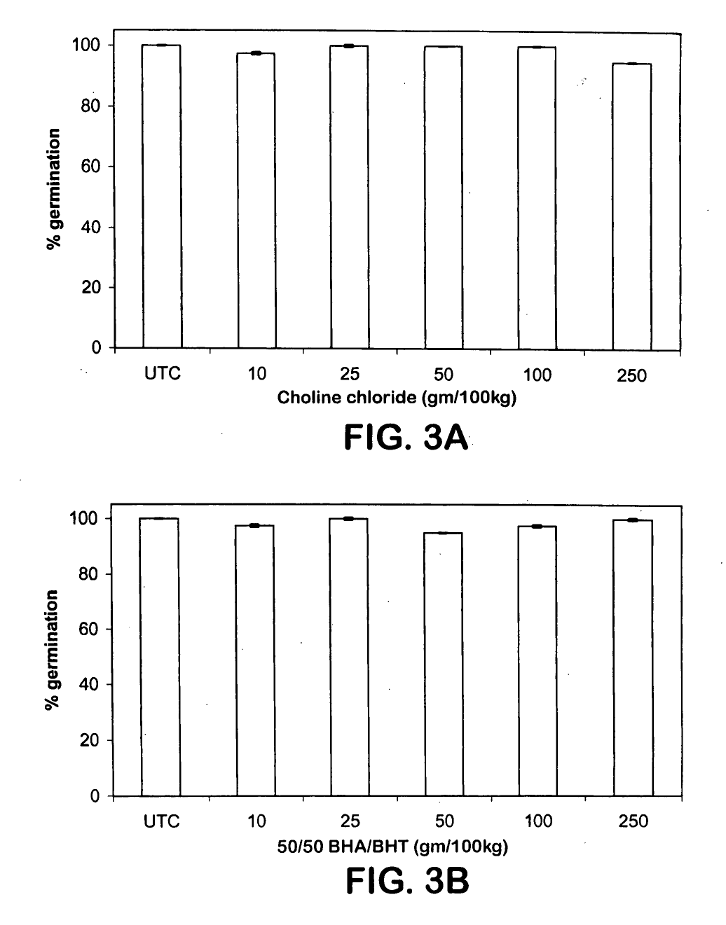

Samples of corn seed “A” ((hybrid LH176rr1.4)(LH224 xLH226), lot no. 5107, a hybrid available from Holden's Foundation Seeds, Inc., Williamsburg, Iowa) with a better cold-tolerant rating than corn seed “B” ((hybrid LH195rr2.1xLH210), lot no. NCP228, rated as less cold-tolerant hybrid than hybrid “A”, also available from Holden's Foundation Seeds, Inc., Williamsburg, Iowa), were treated according to the method described above, and with the choline chloride and BHA/BHT antioxidants and the rates shown in Table 1. After treatment, each seed sample was tested for germination in as described in Test 1, Test 2 and Test 3, as described above. The percent germination of corn seeds in the cold condition germination test is shown in FIG. 1A (Hybrid “A”, BHA/BHT treated), FIG. 1B (Hybrid “B”, BHA/BHT treated), FIG. 2A (Hybrid “A”, choline chloride treated), FIG. 2B (Hybrid “B”, choline chloride treated). The percent germination in optimum (warm) condition germination tests for Hybrid “A” seeds treated with choline chloride is shown in FIG. 3A, and for Hybrid “A” seeds treated with BHA/BHT in FIG. 3B.

The data from the optimum condition germination tests showed no negative effects on germination under normal growing conditions by treatment with either choline chloride or BHA/BHT at any of the treatment levels tested. In the cold condition germination tests with Hybrid “A”, the more cold tolerant hybrid, FIG. 1A showed that treatment with BHA/BHT improved germination rate at lower treatment rates, and 50 g BHA/BHT/100 kg of seed gave 50% germination two days earlier than untreated control, but treatment at levels of 100 g/100 kg and higher appeared to have a negative effect on germination. FIG. 2A showed that choline chloride had substantially the same effect, but with the best rate of 100 g/100 kg of choline chloride providing 50% germination two days earlier than the untreated control.

In the cold condition germination tests for Hybrid “B”, the less cold tolerant hybrid, FIG. 1B showed that treatment with 10 g/100 kg BHA/BHT gave 50% germination four days earlier than untreated control, and FIG. 2B showed that treatment with 50 g and 100 g/100 kg of choline chloride gave 50% germination three days earlier than untreated control. In separate tests, treatment with glycinebetaine gave similar results, and provided three-day earlier germination at 50 g and 100 g/100 kg.

In general, it was believed that the data showed that at certain effective levels, antioxidants, such as BHA/BHT and choline chloride provided improved germination rates under cold stress conditions without negative effects for performance under normal growth conditions.

### Example 3

This example illustrates the effect of seed treatment with antioxidants when young plants have been subjected to a cold shock treatment (such as a cold front coming through a field 2 weeks after planting)

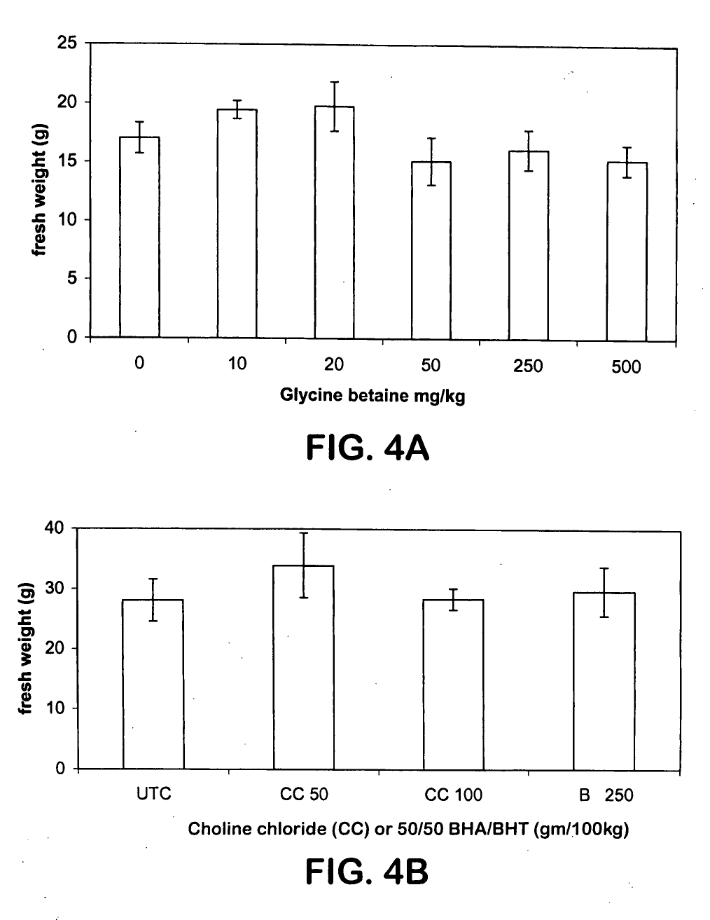

Soybean plantlets (Hybrid AG3303, available from Asgrow Seed Co., St. Louis, Mo.) that were grown in a greenhouse maintained at day/night temperatures of 30° C. day and 25° C. night and with a 12 hour light period. The seed had been treated with different levels of glycinebetaine and were grown in optimum greenhouse conditions until they reached the first trifoliate stage of development. The plants were then moved from the greenhouse and subjected to 22° C./13° C. day/night temperatures for one week in a Conviron growth chamber. After one week the plants were returned to the greenhouse for “recovery” in optimal conditions. The fresh weight accumulation of the plantlets was then measured after 10 days following their return to the greenhouse. FIG. 4A shows that plantlets grown from seed that had been treated with 10-20 g/100 kg of glycinebetaine showed greater fresh weight accumulation following the cold shock. This indicated that the plants grown from treated seed were more cold shock resistant than those having no antioxidant treatment.

Corn plantlets, which were grown from seed (Hybrid LH176rr1.4(LH224xLH226, available from Holden's Foundation Seeds, Inc., Williamsburg, Iowa) that had been treated with various levels of choline chloride and BHA/BHT, as described above, were grown in a greenhouse at 22° C./13° C. day/night temperature until the V1 stage and then they were subjected to 6 days of 18° C./12° C. D/N temperatures with a 16 hour photo period, after which they were returned to the greenhouse for four days prior to measurement of fresh weight accumulation after cold stress. FIG. 4B shows that seeds treated with 250 g/100 kg of BHA/BHT, or 50-100 g/100 kg of choline chloride showed a tendency for greater fresh weight accumulation after cold shock than plantlets grown from untreated seed.

This example shows that plants grown from seed that has been treated with an effective amount of an antioxidant are more resistant to cold injury and recovery more quickly from cold stress than plants grown from untreated seeds.

### Example 4

This example illustrates the effect of seed treatment with antioxidants on the emergence of corn hybrids with different cold-stress tolerance levels.

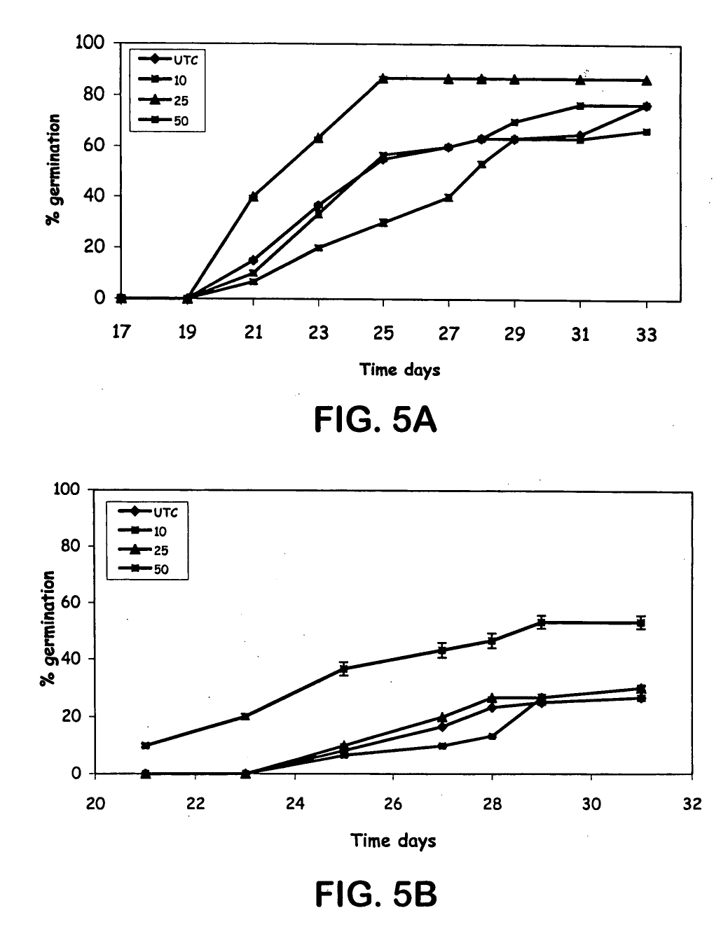

Corn hybrids “A” and “B”, as described above were treated with choline chloride at levels of 0, 10, 25 and 50 g/100 kg of seed. Each batch of seed was tested under 10° C. incubation conditions for emergence from a soil medium as described above. FIG. 5A shows percent emergence versus time for Hybrid “A” (cold tolerant) and FIG. 5B shows percent emergence versus time for Hybrid “B” (cold sensitive). In the experiment with the cold sensitive hybrid, 29 days after seeding, the treatment containing the lowest concentration of choline chloride (10 g/100 kg seed) had reached 50% germination. By the end of the study, 31 days, the untreated control had only reached 25% emergence and was leveling off, presumably with little or no new germination expected. Therefore, a minimum of two days increase in germination time was observed. The first evidence of emergence occurred on day 21 for the 10 g/100 kg treated seed, whereas first evidence of emergence for untreated seed was on day 25.

For the cold tolerant hybrid, 50% emergence was reached on day 22 for the sample treated with 25 g/100 kg seed, a full three days before the untreated control reached the same level of emergence. Accordingly, the data show the improvement in emergence under cold conditions of seed treated with an antioxidant.

### Example 5

This example shows the effect on plant density, vigor and yield of the treatment of soybean seeds with an antioxidant, alone and in combination with a fungicide.

Soybeans (variety AG2103, available from Asgrow Seed Co., St. Louis, Mo.) were treated with an antioxidant, alone or in combination with a pesticide (silthiofam, available from Monsanto Co., St. Louis, Mo., under the trade name LATITUDE®, 11.83% w/w silthiofam) according to the rates shown in Table 2.

TABLE 2
Treatment of soybean seeds with antioxidants alone or in
combination with silthiofam.

| | | Rate of | | |
|---|---|---|---|---|
| | | anti- | | Rate of |
| | | oxidant | | pesticide |
| | | (g/100 kg | | (g/100 kg |
| Sample | Antioxidant | seed) | Pesticide | of seed) |
| Control | 0 | 0 | 0 | 0 |
| silthiofam | 0 | 0 | silthiofam | 25 |
| CC 250 g | choline chloride | 250 | 0 | 0 |
| GB 50 g | glycinebetaine | 50 | 0 | 0 |
| BHA/BHT 10 g | BHA/BHT | 10 | 0 | 0 |
| CC + BHA/ | choline chloride | 250 | 0 | 0 |
| BHT | and | and 10 | | |
| 250 g + 10 g | BHA/BHT | | | |
| silthiofam + CC | choline chloride | 250 | silthiofam | 25 |
| silthiofam + GB | glycinebetaine | 50 | silthiofam | 25 |
| silthiofam + | choline chloride | 250 | silthiofam | 25 |
| CC + BHA/ | and BHA/BHT | and 10 | | |
| BHT | | | | |

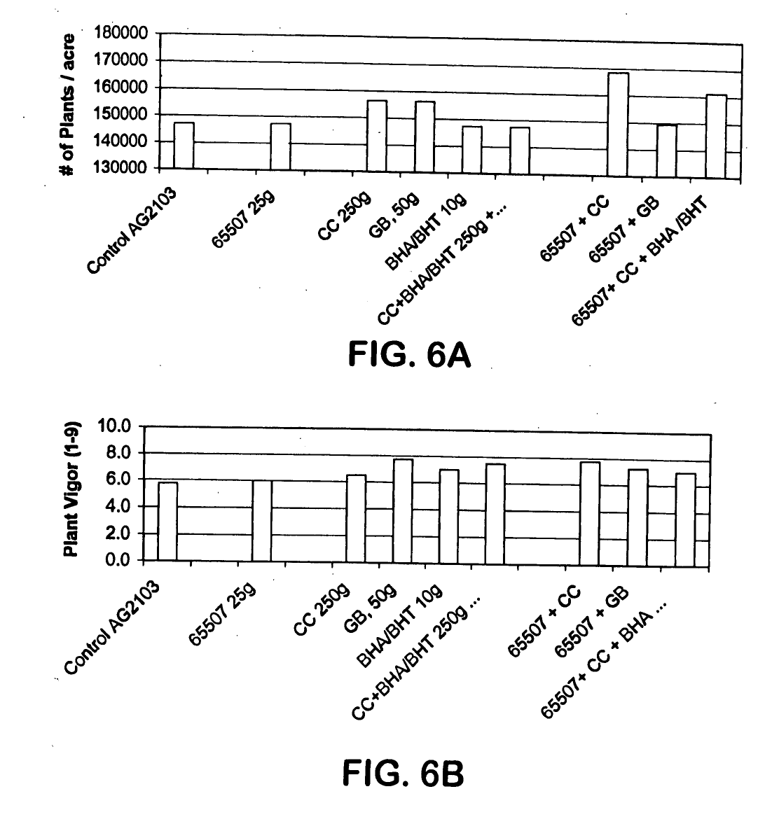

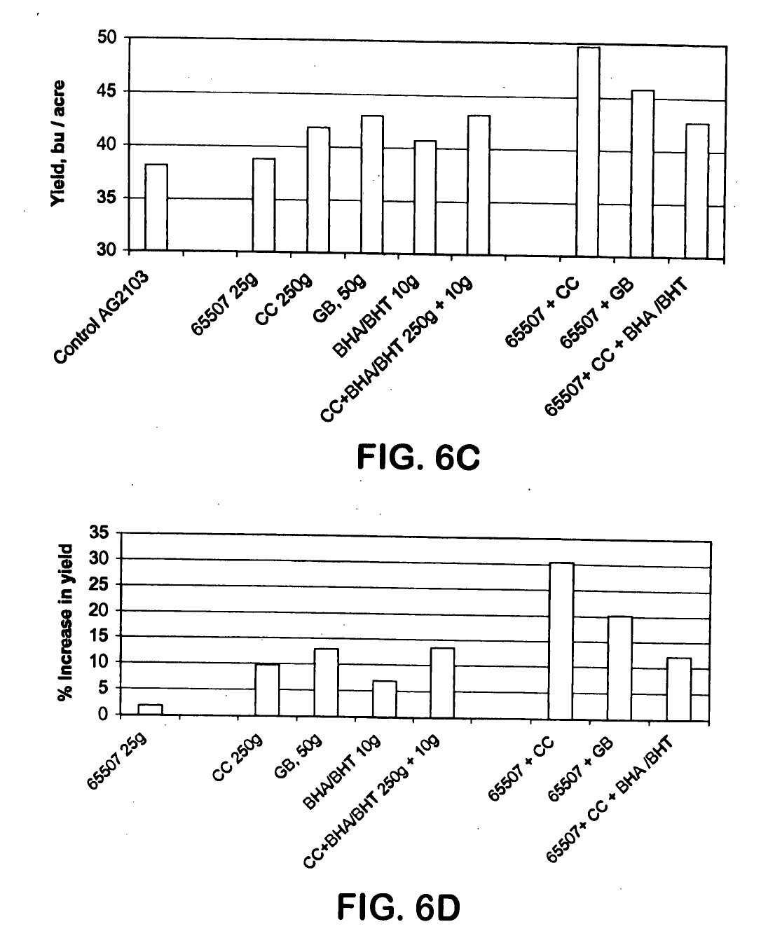

Samples of seed having each type of treatment shown in Table 2 were planted in a controlled field trial in the upper Midwestern United States. Conservation tillage practices were adhered to. During the trial, the plant density (number of plants per acre), the plant vigor (on a standard 0-10 scale, with 0 being worst and 10 best), and the soybean yield (bu/acre) were measured. The results from the test are shown in FIGS. 6A (plant density), 6B (plant vigor), 6C (soybean yield), and 6D (percent increase in soybean yield over an untreated control). From FIGS. 6A and 6B, it was seen that seed treatment with antioxidants alone or in combination with silthiofam gave improved plant populations and improved plant vigor. The addition of silthiofam improved the plant density even further, but had little effect on apparent vigor. For soybean yield, as can be seen in FIGS. 6C and 6D, it was seen that seed treatment with antioxidants alone provided from about 7% to about 13% improvement in yield, while the addition of the pesticide silthiofam in combination with an aintioxidant provided yields that were improved over the untreated control by amounts from about 12% to about 30%. Accordingly, the data show that treatment of soybean seeds with an antioxidant, alone or in combination with a pesticide, improve the plant density, vigor and yield of soybeans.

### Example 6

This example shows the effect on plant density and yield of the treatment of corn seeds with an antioxidant, alone and in combination with a fungicide.

Corn seeds (Hybrid LH176rr1.4(LH224xLH226, available from Holden's Foundation Seeds, Inc., Williamsburg, Iowa) were treated with an antioxidant, alone or in combination with a pesticide (silthiofam, available from Monsanto Co., St. Louis, Mo., under the trade name LATITUDE®, 11.83% w/w silthiofam) according to the rates shown in Table 3.

TABLE 3
Treatment of corn seeds with antioxidants alone or in
combination with silthiofam.

| | | | | Rate of |
|---|---|---|---|---|
| | | Rate of | | pesticide |
| | | antioxidant | | (g/100 kg of |
| Sample | Antioxidant | (g/100 kg seed) | Pesticide | seed) |
| Control | 0 | 0 | 0 | 0 |
| silthiofam | 0 | 0 | silthiofam | 25 |
| CC 250 g | choline | 250 | 0 | 0 |
| | chloride | | | |
| GB 50 g | glycinebetaine | 50 | 0 | 0 |
| BHA/BHT | BHA/BHT | 10 | 0 | 0 |
| 10 g | | | | |
| CC + BHA/ | choline | 250 and 10 | 0 | 0 |
| BHT | chloride and | | | |
| 250 g + 10 g | BHA/BHT | | | |
| silthiofam + | choline | 250 | silthiofam | 25 |
| CC | chloride | | | |
| silthiofam + | glycinebetaine | 50 | silthiofam | 25 |
| GB | | | | |
| silthiofam + | choline | 250 and 10 | silthiofam | 25 |
| CC + BHA/ | chloride and | | | |
| BHT | BHA/BHT | | | |

A seed treating suspension concentrate formulation containing 3% BHA/BHT was prepared via a milling process and corn seeds were treated with the formation at the targeted rates of BHA/BHT. A concentrated choline chloride (35% w/W) liquid formulation was prepared by mixing the active with the solution and corn seed was also treated with this formulation at the targeted rates.

### Preparation of BHA/BHT Suspension Concentrate and Seed Treatment:

BHA (2.34 g), BHT (2.34 g), and CF Clear (4.68 g) polymer emulsion, available from Baker-Underwood, Ames, Iowa, were added to an aqueous solution of EmCOL4500 (1.56 g) in water (145.08 g). The mixture was milled at room temperature for 1.5 hours. Agnique DF 6889 (0.40 g) antifoam, available from Cognis, Cincinnati, Ohio, was added to the mixture and the mixture was milled at room temperature for another 1.5 hours. The mixture became a gray suspension and the particle size was less than 10 micron according to optical microscopy. 140.87 g of the suspension was collected and it contained 3% of the total weight of BHA and BHT in the formulation. The blank was also prepared by mixing water (55.8 g), EMCOL 4500 (0.60 g) surfactant, available from Witco Chemical Co., CF Clear (1.80) and Aqnique DF6889 (0.15 g) and it appeared as a milky emulsion. Corn seeds were treated with this formulation and the blank at the targeted rates of 1, 5, 10, 25, and 50 g/100 kg seed in our small Hege treater. In a typical example corn seed (85 g) was placed in the machine and it was turned on. After 7 seconds, the formulation was added to the rotated machine through a syringe in a second. It was rotated for another 23 seconds and the coated seeds were collected. Since the highest target rate was only 50 g/100 kg seed, no significant loss of the formulation was observed.

### Preparation of Choline Chloride (CC) Formulation:

Choline chloride is highly soluble in water. 35% (w/w) of CC formulation was prepared by dissolving CC (13.125 g) in water (23.36 g) first and then mixing the solution with CF Clear (1.05 g). No polymer precipitated from the formulation and this suggested that CC solution is compatible with this polymer emulsion. The blank was also prepared by mixing water (7.01 g) with CF Clear (1.05 g). In a typical example corn seed (85 g) was placed in the machine and it was turned on. After 7 seconds, the formulation was added to the rotated machine through a syringe in a second. It was rotated for another 23 seconds and the coated seeds were collected. The concentration of CC in the formulation was very high, only small amount of the formulation was thus added to the seed even at a high target rate of 450 g/100 kg seed and no significant loss of the formulation was observed.

### Field Trial Testing of Treated Seed:

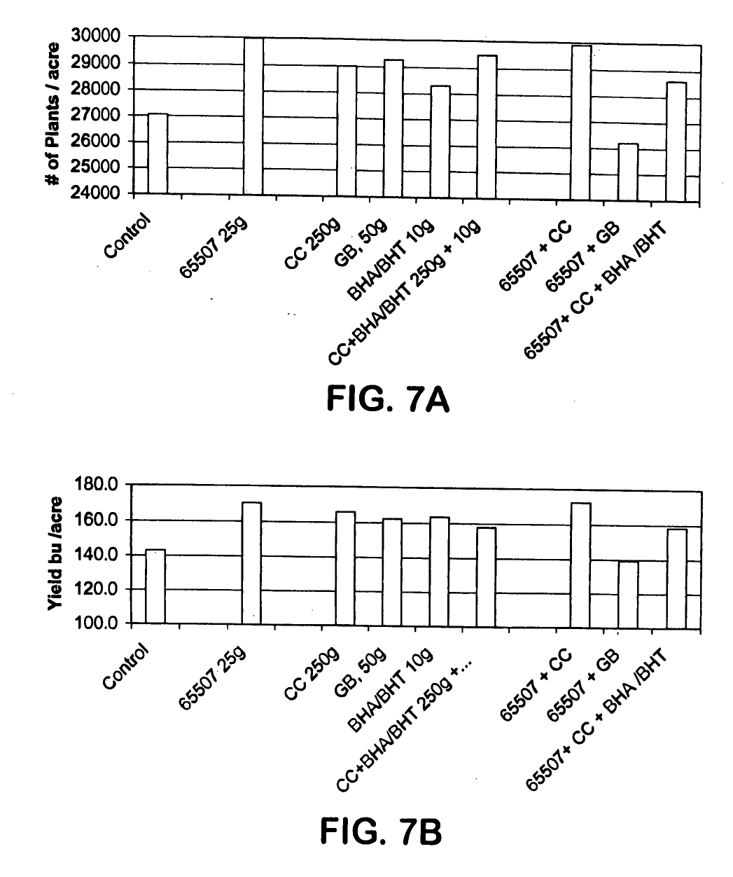

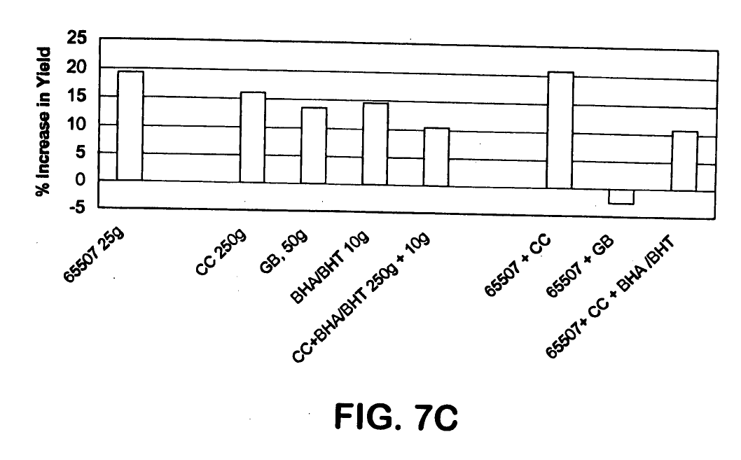

Samples of seed having each type of treatment shown in Table 3 were planted in a controlled field trial in the upper Midwestern United States. Conservation tillage practices were adhered to and planting was accomplished during the first 10% of the crop that got planted in that area. During the trial, the plant density (number of plants per acre) and the corn yield (bu/acre) were measured. The results from the test are shown in FIGS. 7A (plant density), 7B (corn yield), and 7C (percent increase in corn yield over an untreated control). From FIG. 7A, it was seen that seed treatment with silthiofam alone provided a significant improvement in corn plant density, and that antioxidants alone or in combination with silthiofam also gave improved plant populations. For corn yield, as can be seen in FIGS. 7B and 7C, it was seen that seed treatment with silthiofam alone provided almost a 20% yield improvement, while treatment with antioxidants alone improved yield from about 10% to about 16%. The combination of antioxidant with silthiofam varied according to the type of antioxidant, with silthiofam and choline chloride providing over 20% yield improvement, silthiofam and BHA/BHT providing over 10% improvement, and silthiofam plus glycinebetaine even providing a slight yield decrease. For corn, it is believed that the data show that treatment of corn seeds with an antioxidant, alone or in combination with a pesticide, improve the plant density and yield of corn.

In particular, data from field trials shows that the BHA/BHT combination is effective in providing improved emergence in cold conditions at seed treatment rates between 10 g to 25 g/100 Kg. Best results are observed at 10 g/100 Kg rate and negative effect is seen at rates higher than 25 g/100 Kg. However, it is believed that rates lower than 10 g/100 kg should also be effective. Seed treatment with choline chloride has been demonstrated at rates raging from 25 g/100 Kg to 250 g/100 Kg and the emergence is found to increase with the increase of the rate of use of choline chloride.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description and shown in the accompanying drawings shall be interpreted as illustrative and not in a limiting sense. In addition, it should be understood that aspects of the various embodiments may be interchanged both in whole or in part.

1. A method of improving the health and/or yield of a plant that is grown from plant propagation material, the method comprising treating the plant or the plant propagation material with an antioxidant that is selected from the group consisting of glycinebetaine, choline chloride, 2(3)-tert-butyl-4-hydroxyanisole (BHA), butylated hydroxy toluene (BHT), and mixtures thereof, and a fungicide, wherein the amount of the antioxidant and the amount of the fungicide together are effective to improve the health and/or the yield of the plant.

2-6. (canceled)

7. The method according to claim 1, wherein the plant propagation material is a seed and wherein the amount of the antioxidant that is used to treat the plant seed is within the range of from about 5 g to about 400 g/100 kg of the seed.

8. The method according to claim 1, wherein the amount of the fungicide that is used to treat the plant seed is within the range of from about 25 g to about 400 g/100 kg of the seed.

9-25. (canceled)

26. The method according to claim 1, wherein the fungicide is selected from the group consisting of tebuconazole, simeconazole, fludioxonil, fluquinconazole, difenoconazole, 4,5-dimethyl-N-(2-propenyl)-2-(trimethylsilyl)-3-thiophenecarboxamide (silthiofam), hexaconazole, etaconazole, propiconazole, triticonazole, flutriafol, epoxiconazole, fenbuconazole, bromuconazole, penconazole, imazalil, tetraconazole, flusilazole, metconazole, diniconazole, myclobutanil, triadimenol, bitertanol, pyremethanil, cyprodinil, tridemorph, fenpropimorph, kresoxim-methyl, azoxystrobin, ZEN90160, fenpiclonil, benalaxyl, furalaxyl, metalaxyl, R-metalaxyl, orfurace, oxadixyl, carboxin, prochloraz, trifulmizole, pyrifenox, acibenzolar-5-methyl, chlorothalonil, cymoaxnil, dimethomorph, famoxadone, quinoxyfen, fenpropidine, spiroxamine, triazoxide, BAS50001F, hymexazole, pencycuron, fenamidone, guazatine, and cyproconazole.

27-28. (canceled)

29. The method according to claim 1, wherein the treatment comprises contacting the plant propagation material with the antioxidant and the fungicide prior to planting the seed.

30. The method according to claim 1, wherein the treatment comprises contacting the plant propagation with at least one of the antioxidant and the fungicide prior to planting the seed and contacting the seed with the other of the antioxidant and the fungic after planting the seed.

31. The method according to claim 1, wherein the plant or plant propagation material comprises a transgenic event that provides resistance to a herbicide and the plant or plant propagation material is treated with the herbicide.

32. The method according to claim 1, wherein the plant or plant propagation material comprises a transgenic event providing resistance against glyphosate and the plant or plant propagation material is treated with glyphosate.

33. The method according to claim 1, wherein the plant or plant propagation material comprises a transgenic event providing resistance against imidazolinone and the plant or plant propagation material is treated with imidazolinone.

34. A composition for treating a plant seed, the composition comprising an antioxidant that is selected from the group consisting of glycinebetaine, choline chloride, 2(3)-tert-butyl-4-hydroxyanisole (BHA), butylated hydroxy toluene (BHT), and mixtures thereof, and a fungicide.

35-53. (canceled)

54. The composition according to claim 34, wherein the fungicide is selected from the group consisting of tebuconazole, simeconazole, fludioxonil, fluquinconazole, difenoconazole, 4,5-dimethyl-N-(2-propenyl)-2-(trimethylsilyl)-3-thiophenecarboxamide (silthiopham), hexaconazole, etaconazole, propiconazole, triticonazole, flutriafol, epoxiconazole, fenbuconazole, bromuconazole, penconazole, imazalil, tetraconazole, flusilazole, metconazole, diniconazole, myclobutanil, triadimenol, bitertanol, pyremethanil, cyprodinil, tridemorph, fenpropimorph, kresoxim-methyl, azoxystrobin, ZEN90160, fenpiclonil, benalaxyl, furalaxyl, metalaxyl, R-metalaxyl, orfurace, oxadixyl, carboxin, prochloraz, trifulmizole, pyrifenox, acibenzolar-5-methyl, chlorothalonil, cymoaxnil, dimethomorph, famoxadone, quinoxyfen, fenpropidine, spiroxamine, triazoxide, BAS50001F, hymexazole, pencycuron, fenamidone, guazatine, and cyproconazole.

55-64. (canceled)

65. Treated plant propagation material, comprising:
a) plant propagation material; including,
b) an antioxidant that is selected from the group consisting of glycinebetaine, choline chloride, 2(3)-tert-butyl-4-hydroxyanisole (BHA), butylated hydroxy toluene (BHT), and mixtures thereof, that has been added to the plant propagation material; and
c) a fungicide.

66-67. (canceled)

68. The treated plant propagation material according to claim 65, wherein the plant propagation material is of a plant that is selected from the group consisting of corn, peanut, canola/rapeseed, soybean, curcubits, cotton, rice, sorghum, sugar beet, wheat, barley, rye, sunflower, tomato, sugarcane, tobacco, oats, potato, vegetable, and leaf crops.

69. The plant propagation material according to claim 68, wherein the plant propagation material is a seed of a plant that is selected from the group consisting of corn, soybeans, and cotton.

70. The seed according to claim 68, wherein the seed is a soybean seed.

71. The plant propagation material according to claim 65, wherein the plant propagation material comprises a transgenic event.

72. The plant propagation material according to claim 71, wherein the transgenic event is one that provides resistance to a herbicide.

73. The method according to claim 1, wherein the antioxidant comprises glycinebetaine.

74. The method according to claim 1, wherein the antioxidant comprises choline chloride.

75. The method according to claim 1, wherein the antioxidant comprises 2(3)-tert-butyl-4-hydroxyanisole (BHA) and/or butylated hydroxy toluene (BHT).
